(19) United States
(12) Patent Application Publication
(10) Pub. No.: US 2024/0197165 A1
(43) Pub. Date: Jun. 20, 2024

(54) METHOD OF AND SYSTEM FOR OBESITY TREATMENT USING LAPAROSCOPICALLY-GUIDED 3D-STEREOSCOPIC AND IR-THERMOGRAPHIC INTRA-ABDOMINAL VISCERAL FAT ASPIRATION, SUPPORTED BY REAL-TIME CYTOKINE SENSING AND PROFILING AND AUGMENTED-REALITY (AR) DISPLAY AND VISUAL SAMPLE TAGGING

(71) Applicant: Rocin Laboratories, Inc., West Palm Beach, FL (US)

(72) Inventor: Robert L. Cucin, West Palm Beach, FL (US)

(73) Assignee: Rocin Laboratories, Inc., West Palm Beach, FL (US)

(21) Appl. No.: 18/068,522

(22) Filed: Dec. 20, 2022

(51) Int. Cl. A61B 1/313; A61B 1/00; A61B 1/05; A61B 17/00; A61B 17/34; A61B 34/30; A61B 90/00

(52) U.S. Cl. CPC A61B 1/3132; A61B 1/00193; A61B 1/05; A61B 17/00234; A61B 17/3421; A61B 34/30; A61B 90/361; A61B 90/37

(57) ABSTRACT

A 3D-stereoscopic IR-thermographic intra-abdominal visceral fat aspiration system and method employing a powered visceral fat aspiration instrument held by a surgeon or surgical robot, and having an electro-cauterizing, irrigating and photo-ablating twin-cannula assembly for use in safely removing visceral fat from the mesenteric region of a patient, through one or more small incisions in the patient's body, while supporting real-time cytokine-sensing and profiling with Augmented-Reality (AR) guidance and visual sample tagging.

### BACKGROUND OF THE INVENTION

### Field of the Invention

The present invention relates to a novel way of and means for treating abdominal obesity, metabolic syndrome and Type II diabetes mellitus in human patients, and improved ways of performing in situ fat tissue analysis and removal for diverse methods of treatment.

### Brief Description of the State of Knowledge in the Art

Applicant's U.S. Pat. Nos. 11,259,862; 9,925,314; and 7,112,200 teach in great detail how it is now possible to safely remove, on a out-patient basis, significant amounts of visceral fat deposits from within the mesentery region of a patient, suffering from metabolic syndrome and/or Type II Diabetes, caused by obesity.

U.S. Pat. No. 9,744,274 teaches using a twin-cannula fat aspiration instrument, provided with RF electro-cautery electrodes and a sump-like pumping action with irrigation fluid at the distal portion of the cannula. U.S. Pat. No. 9,744,274 also teaches the use of an in-line tissue collection device, that allows for the collection of aspirated visceral fat tissue for analysis and reinsertion into the patient's body.

Using such techniques and technologies, it is now possible to safely remove visceral fat from a patient's mesentery region, and quickly transform the metabolic state of the patient on an out-patient basis by removing metabolically-active visceral fat tissue from the patient's body. This, in turn, removes the source of cytokines that inhibit insulin absorption at the cellular level, and deregulate satiation and appetite control within the patient suffering from metabolic syndrome and/or Type II diabetes.

To support this method of treatment, U.S. Pat. No. 9,744,274 teaches the use of a manually-operated fat tissue sampling and collection device installed in-line between the fat tissue aspiration instrument and the vacuum source connected to the aspiration instrument. By sampling and collecting visceral fat tissue samples from particular regions with the patient's body, and apply rapid on-site lab testing, it is possible to determine which regions of visceral fat have high metabolic activity, relative to sampled fat deposits, and are preferred for removal over other deposits within the patient's abdomen, during a course of obesity treatment. While this method is promising in principle, there remains a great need for improvements in terms of speed and feedback from the laboratory handling fat tissue metabolic activity analysis.

Cytokines are soluble proteins secreted by immune cells that act as molecular messengers relaying instructions and mediating various functions performed by the cellular counterparts of the immune system, by means of a synchronized cascade of signaling pathways. Cytokines are secreted in response to an inflammatory stimulus by nearly all nucleated cells, particularly immune cells or leucocytes. While structurally versatile, cytokines are grouped together based on their biological functions, which are similar in principle and often orchestrated in an interdependent manner. The primary cytokines associated with obesity and metabolic syndrome are: Resistin—contributing to D2M; Angiotensin—contributing to high blood pressure; Tumor Necrosis Factor (TNF-alpha)—contributing to inflammation; Interleukin-6—contributing to inflammation; Adiponectin—contributing to narrowing of arteries; and insufficient or antibodies to Leptin (satiety hormone) contributing to deregulation of appetite control.

Applicant's U.S. Pat. No. 9,744,274 teaches that visceral fat within the mesentery of a patient functions as cytokine factory associated with metabolic syndrome, obesity and type-II diabetes, and that during visceral fat tissue aspiration operations, visceral fat deposits can be (i) aspirated using Applicant's endoscopic/laparoscopic fat aspiration system, (ii) collected in Applicant's in-line tissue sampling and collection device, and (iii) analyzed in the laboratory to provide the surgeon feedback on metabolic activity of specific visceral fat deposits within the patient's body that should be targeted for removal.

Clearly, cytokine profiling can provide valuable information for diagnosing such diseases and disorders, monitoring their progression, as well as assessing the efficacy of therapeutic treatment.

Toward this general goal of cytokine profiling in the field of medicine, there has been immense interest in the development of ultrasensitive quantitative detection techniques for cytokines, which may involve the use of technologies from various scientific disciplines, such as immunology, electrochemistry, photometry, nanotechnology and electronics. The article titled “Electrochemical Biosensors for Cytokine Profiling: Recent Advancements and Possibilities in the Near Future” by Nirmita Dutta et al, published in Biosensors 2021, 11, 94, provides a review on electrochemical biosensors that are being considered extremely promising for routine clinical testing in the near future.

While there is great promise that the removal of visceral fat in the mesenteric region of human patients stands to ameliorate the metabolic syndrome and abdominal obesity, and reduce morbidity due to obesity, there is a great need in the art for new and improved methods of and apparatus for safely removing visceral fat in human patients, without employing conventional direct surgical excision techniques, and exposing patients to the high risk of vascular injury with concomitant bleeding and vascular compromise of the intestine, associated with conventional surgical procedures and apparatus.

### Objects of the Present Invention

Accordingly, it is a primary object of the present invention to provide a new and improved method of and apparatus for safely removing soft tissue, such as mesenteric fat, in human patients to ameliorate chronic conditions, such as metabolic syndrome or abdominal obesity, while avoiding the shortcomings and drawbacks of conventional surgical procedures and apparatus.

Another object of the present invention is to provide such apparatus in the form of a 3D-stereoscopic, IR-thermographic laparoscopically-guided intra-abdominal visceral fat aspiration system and method employing a powered hand-supportable fat aspiration instrument held by a surgeon, or surgical robot, and having an electro-cauterizing, irrigating and photo-ablating twin-cannula assembly for use in safely removing visceral fat from the mesenteric region of a patient, through one or more small incisions in the patient's body, while supporting real-time cytokine-sensing and profiling with Augmented-Reality (AR) guidance and visual sample tagging.

Another object of the present invention is to provide a new and improved method of and system for visceral fat tissue removal and obesity treatment using 3D-stereoscopic and IR-thermographic laparoscopically-guided intra-abdominal visceral fat aspiration, with real-time cytokine-sensing and profiling with augmented-reality (AR) guidance and visual sample tagging.

Another object of the present invention is to provide a new and improved method of 3D-stereoscopic IR-thermographic laparoscopically-guided intra-abdominal visceral fat aspiration using apparatus that enable safe removal of visceral fat tissue from the mesenteric region of a patient, on an out-patient basis, while supporting real-time cytokine-sensing and profiling with augmented-reality (AR) guidance and visual sample tagging.

Another object of the present invention is to provide a new and improved method of and apparatus for performing laparoscopic mesenteric visceral fat aspiration for ameliorating the metabolic syndrome, or abdominal obesity of the patient.

Another object of the present invention is to provide such a method comprising the steps of inserting a 3D-stereoscopic IR-thermographic laparoscopic imaging instrument, along with an electro-cauterizing visceral fat aspiration instrument into the mesenteric region of a patient, for the purpose of safely removing visceral fat so as to ameliorate the metabolic syndrome, or abdominal obesity of the patient.

Another object of the present invention is to provide a novel method of 3D-stereoscopic/IR-thermographic imaging system for laparoscopically-guiding intra-abdominal visceral fat aspiration, for the purpose of safely removing metabolically-active visceral fat within the mesentery region of a patient's abdomen, as part of a safe and effective method of treating obesity, metabolic syndrome and/or type-II diabetes.

A further object of the present invention is to provide a novel method of treatment for and amelioration of type II diabetes mellitus, and produce a favorable effect on metabolism including increasing insulin sensitivity, lowering fasting blood sugar, a lowering of blood pressure (particularly diastolic), improving the lipid profile (lowered cholesterol, raising HDL, lowered triglycerides, lowering serum adipocytokine (Leptin) and inflammatory markers (TNF-α=tumor necrosis factor, resistin, IL-6 and IL-9), and by doing so, effecting a decrease in insulin resistance and reduce the risk of coronary artery disease associated with metabolic syndrome.

Another object of the present invention is to provide an improved method of treating type II diabetes by way of selected removal of visceral fat cells and components contained therein (e.g. fat, adipocytokine (Leptin) and inflammatory markers (TNF-α=tumor necrosis factor, resistin, IL-6 and IL-9), to improve the sensitivity of tissue cells to insulin.

Another object of the present invention is to provide a new and improved coaxially-driven visceral fat aspiration instrument employing a twin-cannula assembly that performs visceral fat aspiration operations in a mechanically assisted manner.

Another object of the present invention is to provide a new and improved visceral fat aspiration instrument system which comprises a hand-supportable fat aspiration instrument and a twin-type cannula assembly, which can be driven by pressurized air or electricity, and in which the cannula assembly is disposable.

Another object of the present invention is to provide an improved method of performing visceral fat aspiration, in which one of the cannulas of the cannula assembly is automatically reciprocated back and forth relative to the hand-holdable housing, to permit increased control over the area of subcutaneous tissue where fatty and other soft tissue is to be aspirated.

Another object of the present invention is to provide a power-assisted visceral fat aspiration instrument system, and 3D-stereoscopic/IR-thermographic laparoscopic instrumentation, provided with an in-line cytokine analysis and detection system for supporting real-time detection and measurement of cytokine concentrations contained in aspirated fat safely removed from a patient's mesentery region during visceral fat tissue lipectomy operations performed on an outpatient basis.

Another object of the present invention is to provide such an air-powered tissue-aspiration instrument system, wherein an intelligent instrument controller is used to supply air-power to the inner cannula reciprocation mechanism within the hand-supportable instrument, while communicating control signals between the instrument and its intelligent controller.

Another object of the present invention is to provide a visceral fat tissue aspiration instrument system having a real-time cytokine-sensing and profiling module employing an impedance sensor with sensor electrodes installed within an array of microchannels, through which a specimen is transported for analysis and profiling, wherein the impedance sensor continuously measures the impedance change between the sensor electrodes arising from the specific binding of the target protein to the cytokine antibody already present within the microchannels.

Another object of the present invention is to provide such a visceral fat tissue aspiration instrument system, wherein the specific binding of the target protein to the cytokine antibody affects ion transport inside the microchannels, resulting in a change in impedance between the two electrodes, and as a result, real-time monitoring of both the antibody attachment process and target protein binding can be achieved by continuously capturing this change in impedance.

Another object of the present invention is to provide a new and improved AR/VR-supported surgical system for use in remote metaverse-type operating environments, wherein surgeons maybe remotely situated from patients undergoing endoscopic visceral lipectomy procedures, and the like, on an outpatient basis as part of a safe and effective obesity treatment methodology and program.

Another object of the present invention is to provide such AR/VR-supported surgical system for use in remote metaverse environments configured for the purpose of teaching, conducting workshops and recording sessions in the metaverse using AR-supported methods of the present invention.

Another object of the present invention is to provide a new and improved AR-supported surgical system enabling real-time in-flow detection of other specific biomarkers for cancer (e.g. PSA for prostate cancer, CEA for colon cancer, and CA125 for ovarian cancer) so as to provide surgeons with quick and instant feedback regarding the status of having attained clear margins of operative resection during surgical procedures.

Another object of the present invention is to provide a new and improved microfabricated microchannel impedance-based protein-detection sensor chip for rapid-sample-to-answer, label-free detection of cytokines, other biomarkers, and proteins of interest, supporting real-time in vivo quantification thereof, by way of continuous measurement of the electrical impedance change between the gold sensor electrode surfaces inside the microchannel arising from the specific binding of the target protein to the cytokine antibody already present within the microchannels.

Another object of the present invention is to provide a new and improved microfabricated microchannel impedance-based protein-detection sensor chip, wherein specific binding of the target protein to the cytokine antibody affects ion transport inside the microchannels, resulting in a change in impedance between the two electrodes, thereby enabling real-time monitoring of both the antibody attachment process and target protein binding by continuously capturing the change in electrical impedance across the microchannel.

Another object of the present invention is to provide a new and improved bariatric surgery operating room provided with a laparoscopically-guided 3D-stereoscopic and IR-thermographic intra-abdominal visceral fat aspiration system, supported by real-time cytokine-sensing and profiling with augmented-reality (AR) guidance and visual sample tagging, and configured and operational for treating metabolic syndrome in human patients by non-invasively and safely removing visceral fat tissue deposits from within the mesenteric region thereof on an ambulatory basis

Another object of the present invention is to provide a new and improved method of and system for visceral fat tissue removal and obesity treatment using laparoscopically-guided 3D-stereoscopic and IR-thermographic intra-abdominal visceral fat aspiration, with real-time cytokine-sensing and profiling with augmented-reality (AR) guidance and visual sample tagging.

Another object of the present invention is to provide a new and improved method of laparoscopically-guided 3D-stereoscopic IR-thermographic intra-abdominal visceral fat aspiration using apparatus that enable safe removal of visceral fat tissue from the mesenteric region of a patient, on an out-patient basis, while supporting real-time cytokine-sensing and profiling with augmented-reality (AR) guidance and visual sample tagging.

These and other objects of the present invention will be described in greater detail hereinafter in the claims to invention appended hereto.

### BRIEF DESCRIPTION OF THE DRAWINGS

The above Objects of the Present Invention will be more fully understood when taken in conjunction with the following Figure Drawings, wherein like elements are indicated by like reference numbers, wherein:

FIGS. 1A and 1B show a surgical operating room designed, constructed and equipped in accordance the principles of the present invention, for supporting safe non-invasive removal of visceral fat tissue from a patient's mesentery region on an out-patient basis using real-time methods and apparatus adapted for visceral fat removal, processing and analysis, and indexing, so as to support various methods of treatment of obesity in accordance with the principles of the present invention;

FIG. 1C shows a surgeon wearing AR/VR headgear to insert the surgeon into a virtual world, or metaverse, and interact with his or her surgical workspace (i.e. field of view) in an operating room while aspirating and sampling visceral fat tissue and viewing AR-display images with IR thermographic color-encoding and cytokine-concentration information displays provided in a heads-up manner, for the benefit and advantage of the surgeon during the practice of the tissue aspiration operation according the to present invention;

FIG. 2 shows the surgical operating room arranged according to the present invention shown in FIGS. 1A and 1B, and illustrating the manually-operated laparoscopically-guided power-assisted visceral fat aspiration system of the present invention being used to surgically treat an obese patient in the operating room while undergoing a mesenteric visceral fat aspiration procedure carried out using the same, supported by 3D AR Stereoscopic Image Compositing and Real-Time Cytokine Analysis and Detection Methods according to the present invention;

FIG. 3 shows the components of the Manually-Operated 3D-Stereoscopic IR-Thermo-Laparoscopic Visceral Fat Removal System and Method of the present invention shown in FIGS. 1A, 1B and 2, wherein the System comprises a 3D stereoscopic endoscopic camera system with IR image sensors, a powered twin-cannula tissue aspiration instrument, a fat tissue vacuum and collection tank, a real-time cytokine detection and analysis subsystem, an augmented-reality (AR) video processor, a 3D stereoscopic display system and headset, with audio transducers, and left and right visual channel processing module with IR encoding, an irrigation fluid source, a tissue aspiration controller, and a real-time multi-channel cytokine detection chip comprising a multiplexer at its input fluid port, six sensor channels through which heated aspirated fat samples flow toward the output fluid port under controlled pressure, while being electrically analyzed across the sensing channel, a real-time electrical impedance (Z) analyzer and process module, a real-time cytokine profiling and formatting module, interfaced with the AR video processor of the system, for displaying detected concentrations of specific cytokines in each tagged sample of visceral fat sampled and aspirated from a particular patient's abdominal region;

FIG. 3A shows a schematic block functional diagram of a multi-microchannel (i.e. sensor) formed on the real-time cytokine detection and analysis chip of the present invention, comprising a number of subcomponents, namely, (i) a micro-fluidic detection chip module, wherein each sensing channel has an input micro-fluidic inflow gate operably connected to the powered aspiration instrument inserted and guided within the patient, and also connected to a micro-fluidic flow detection channel that is connected to an output microfluidic outflow gate, that is operably connected to a flow channel vacuum source, (ii) a flow channel flushing agent module in fluid communication with each micro-fluidic flow detection channel, for flushing the channel with an appropriate flushing agent under the control of the system controller, (iii) a flow channel liquid cytokine binding agent module in fluid communication with each micro-fluidic flow detection channel, for supplying the channel with an appropriate cytokine binding agent under the control of the system controller, suited for the particular cytokine to be detected within this specific flow channel on the chip, (iv) a ultrasonic-based electrode cleaning module in fluid communication with each micro-fluidic flow detection channel, for ultrasonically cleaning the channel with ultrasonic energy and applied fluid under the control of the system controller, (v) a flow channel impedance measuring module in fluid communication with each micro-fluidic flow detection channel, for electrically measuring the impedance (Z) characteristics of the fat sample flowing within the sensing channel under the control of the system controller, (vi) an impedance data processing module in communication with the flow channel impedance measuring circuit module, for processing the electrical data collected by the impedance measuring circuit under the control of the system controller, (vii) a cytokine detection module in communication with the impedance data processor for processing the data and detecting the type and quantity (i.e. concentration) of cytokine molecules in the specific flow sensing channel aboard the chip device, for providing digital output to the AR display controller while it is receiving captured digital image frames and indices during the time of fat sampling, and compositing the data input to generate a composite video output for visual display on the 3D stereoscopic IR display system;

FIG. 3B is a flow chart describing the primary steps of a method of real-time cytokine detection by impedance measurement across each micro-fluidic channel formed on a cytokine detection chip, through which liquid visceral fat tissue samples flow during real-time cytokine detection and analysis carried out as part of a non-invasive method of visceral-fat removal from patients suffering from metabolic syndrome, comprising the steps of (a) in response to a cytokine detection request, automatically initiating a flush cycle across a flow sensing channel (i.e. microchannel), initiate electrode cleaning (i.e. ultrasonic cleaning) across the sensing flow channel, applying cytokine binding agents to the flow sensing channel surfaces, and making electrical impedance measurements across the prepared flow sensing channel during a pre-detection flow channel calibration procedure, (b) transporting aspirated visceral fat tissue sample across a prepared flow sensing channel, (c) making electrical measurements across the prepared flow sensing channel during a pre-detection flow channel calibration procedure.

FIG. 3C is a schematic illustration of the real-time cytokine detection system of the present invention supporting impedance measurement across each micro-fluidic flow channel formed on the cytokine detection chip, through which liquid visceral fat tissue samples flow during real-time cytokine detection and analysis for processing in the micro-fluidic flow channels and output from the chip, wherein stereoscopic digital video images of sampled fat tissue region undergoing automated cytokine detection are provided for input processing, and AR-image encoding of detected cytokine concentrations, to produce as output, AR-based digital video images containing cytokine concentration measures for digital display;

FIG. 4 is a perspective view of the manually-operated power-assisted fat aspiration instrument system shown deployed in the surgical operating room environment of FIGS. 1A, 1B, 2, 3, and 3A-3C having a twin-cannula assembly supporting three-functions (i.e. tumescent infusion, electro-cautery and YAG-based photo-ablative laser illumination and delivery) about the aspiration aperture during visceral fat aspiration operations;

FIG. 5A is a first perspective view of a Robotically-Controlled 3D Stereoscopic IR-Thermographic Laparoscopic Surgical (Robotic) System of the present invention, configured with the powered fat tissue aspiration cannula instrument of the present invention, and the real-time cytokine detection and analysis subsystem, illustrated in FIGS. 1A, 1B, 2, 3A-3C and 4;

FIG. 5A1 is a first perspective view of the hand-controlled interface subsystem employed in the Robotically-Controlled 3D-Stereoscopic IR Thermographic Laparoscopic Surgical Robot System of the present invention, shown in FIG. 5A;

FIG. 5A2 is a second perspective view of the hand-controlled interface subsystem employed in the Robotically-Controlled 3D-Stereoscopic IR-Thermographic Laparoscopic Surgical Robotic System of the present invention, shown in FIG. 5A;

FIG. 5B is a second perspective view of the Robotically-Controlled 3D-Stereoscopic IR-Thermographic Laparoscopic Surgical Robotic System of the present invention shown in FIG. 5A, configured with the powered fat tissue aspiration cannula instrument of the present invention held and controlled by a robotic navigation subsystem that handles the other surgical instruments depicted in FIGS. 2 and 3;

FIG. 5C is a third perspective view of the Robotically-Controlled 3D-Stereoscopic IR-Thermographic Laparoscopic Surgical Robotic System of the present invention shown in FIGS. 5A and 5B, configured with the powered fat tissue aspiration cannula instrument of the present invention held and controlled by a robotic navigation subsystem that handles the other surgical instruments depicted in FIGS. 2 and 3;

FIG. 6A is a plan view of an operating room layout showing the Robotically-Controlled 3D Stereoscopic-IR Thermographic Laparoscopic Surgical Robotic System of FIGS. 5A and 5B, configured with the powered fat tissue aspiration cannula instrument of the present invention held and controlled by a robotic navigation subsystem that handles the other surgical instruments depicted in FIGS. 2 and 3;

FIG. 6B is a schematic illustration of a global AR-VR metaverse-based surgical system network of the present invention that supports the various systems and subsystems disclosed herein on its internet infrastructure, including the 3D-stereoscopic IR-thermographic endoscopic visceral fat tissue aspiration system, configured with real-time cytokine detection and AR-based surgical display capabilities of the type and kind described herein;

FIG. 7 is a schematic illustration of the operating room layout of FIG. 6, showing the Robotically-Assisted 3D-Stereoscopic IR-Thermographic Laparoscopic Surgical (Robotic) System of FIGS. 5A and 5B, configured with the powered fat tissue aspiration cannula instrument of the present invention held and controlled by a robotic navigation subsystem that handles the other surgical instruments depicted in FIGS. 2 and 3;

FIG. 8 is a detailed system block diagram showing the components of the 3D-Stereoscopic IR-Thermographic Laparoscopic Visceral Fat Removal System and Method of the present invention shown in FIGS. 5A, 5B, 5C, 6 and 7, also supporting (i) a 3D stereoscopic endoscopic digital camera system with IR image sensors, (ii) a powered twin-cannula tissue aspiration instrument system, and (iii) a real-time cytokine detection and analysis subsystem, and profiling using augmented-reality (AR) guidance and visual sample tagging methods;

FIG. 9 is a schematic representation of the 3D stereoscopic endoscopic digital camera system with IR image sensors employed in the 3D-Stereoscopic IR-Thermographic Laparoscopic Visceral Fat Removal System shown in FIGS. 5A, 5B, 5C, 6 and 7;

FIG. 10A is a first schematic representation of the 3D stereoscopic endoscopic digital camera system showing the embedding of IR image sensors in at least one channel of the 3D-Stereoscopic IR-Thermo-Laparoscopic Visceral Fat Removal System shown in FIGS. 5A, 5B, 5C, 6 and 7;

FIG. 10B is a second schematic representation of the 3D stereoscopic endoscopic digital camera system showing the embedding of IR image sensors in at least one channel of the 3D-Stereoscopic IR-Thermographic Laparoscopic Visceral Fat Removal System shown in FIGS. 5A, 5B, 5C, 6 and 7;

FIG. 11 is a schematic representation of the 3D stereoscopic endoscopic digital camera system shown in FIGS. 10A and 10B, employed in the 3D-Stereoscopic IR-Thermographic Laparoscopic Visceral Fat Removal System shown in FIGS. 1A, 1B, 2, 3 and 5A, 5B, 5C, 6 and 7, wherein many different kinds of 3D imaging/display techniques can be supported by the 3D stereoscopic endoscopic digital camera system of the present invention, within the 3D Stereo-IR-Thermo-Laparoscopic System for Visceral Fat Removal, in accordance with the principles of the present invention;

FIG. 12 is a flow chart describing the primary steps involved in the method of color-coding pixels captured in the surface-temperature IR-thermographically images of visceral fat tissue within the mesentery region of a patient during surgical treatment according to method of the present invention;

FIG. 13 is a schematic representation of a color-coded image of IR-thermographically imaged visceral fat tissue within the mesentery region of a patient undergoing surgical treatment according to method of the present invention

FIG. 14 is a perspective view of the bipolar electro-cauterizing twin-cannula visceral fat photo-ablation and aspiration instrumentation system of the present invention, depicted in the system of FIG. 1, and shown comprising (i) a hand-supportable fat aspiration instrument having (i) a hand-supportable housing with a stationary tubing connector provided at the rear of the housing and receiving a length of flexible tubing connected to a vacuum source and connecting to the cylindrical cannula base portion guide tube, and a twin tumescent-type cannula assembly having an inner cannula coupled to an electrically-powered cannula drive mechanism disposed within the hand-supportable housing and powered by a source of electrical power, while its stationary outer cannula is releasably connected to the front portion of the hand-supportable housing, and (ii) a system controller for controlling the electro-cautery, irrigation and YAG-laser driven photo-ablative functions supported by the fat aspiration instrument;

FIG. 15A shows an exploded view of the photo-ablative electro-cauterizing fat aspiration instrument of the present invention depicted in FIGS. 14 and 15B, showing its components disassembled;

FIG. 15B is a perspective view of the electromagnetically-powered fat tissue photo-ablation and aspiration instrument shown in FIG. 13, having a twin-cannula assembly supporting three-functions (i.e. tumescent infusion, electro-cautery and YAG-laser photo-ablation) about the aspiration aperture during visceral fat aspiration operations;

FIG. 16A is a first partially cut-away perspective view of the distal (tip) portion of the outer cannula component of the twin-cannula assembly of the present invention, illustrating its optical fiber carrying the YAG-laser photonic-energy laser beam to the field about the outer aspiration aperture to support photo-ablative operations on visual fat tissue present within the field, and an irrigation channel for conducting irrigation fluid to the bullet tip area of the outer cannula where aspiration and photo-ablative operations are carried out;

FIG. 16B is a plan view of the distal portion of the cannula component of the twin-cannula assembly of the present invention shown in FIGS. 12, 13A, 13B and 14, and illustrating its optical fiber carrying the YAG-laser photonic energy beam to the field about the outer aspiration aperture, and irrigation channel conducting irrigation fluid to the bullet tip area of the outer cannula;

FIG. 17 is a perspective view of the in-line fat sampling and real-time cytokine detection and analysis device of the present invention depicted in FIGS. 3, 4, 7 and 8, and shown operably connected between a vacuum source and the visceral fat tissue aspiration instrument of the present invention, shown in FIGS. 3 and 7;

FIG. 18A is a perspective view of in-line fat sampling and real-time cytokine detection and analysis device of the present invention, depicted in FIGS. 3, 4, 7, 8, and 12; and

FIG. 18B is a cross-sectional view of the in-line fat sampling and real-time cytokine detection and analysis device of the present invention, depicted in FIGS. 17 and 18A; and

FIG. 19 is a schematic representation of a color-coded image of IR-thermographically imaged visceral fat tissue within the mesentery region of a patient undergoing surgical treatment of the present invention, shown with an augmented-reality (AR) display of real-time measurement of the cytokine concentration present in an aspirated visceral fat tissue sample during a time-stamped and specified sampling interval during the surgical treatment procedure of the present invention.

### DETAILED DESCRIPTION OF THE ILLUSTRATIVE EMBODIMENTS OF THE PRESENT INVENTION

Referring to the figures in the accompanying Drawings, the various illustrative embodiments of the present invention will be described in great detail, wherein like elements will be indicated using like reference numerals.

The following U.S. patent applications are owned by Rocin Laboratories, Inc., and incorporated herein by reference in their entirety: U.S. application Ser. No. 13/315,230 filed Dec. 8, 2011 and now U.S. Pat. No. 9,833,279; which is a Continuation of application Ser. No. 12/850,786 filed Aug. 5, 2010 and now U.S. Pat. No. 8,465,471; which is a Continuation-in-Part (CIP) of application Ser. No. 12/462,596 filed Aug. 5, 2009, and application Ser. No. 12/813,067 filed Jun. 10, 2010.

### Overview on Methods of Treatment According to Principles of the Present Invention

In general, the method of treatment according to the present invention involves performing vacuum-assisted aspiration of mesenteric fat from a patient in the intra-abdominal region, using a new and improved minimally-invasive, 3D-stereoscopic/IR-thermographic based “laparoscopic” procedure using the new and improved fat aspiration instruments of the present invention shown in the Drawings and described throughout the Patent Specification, that will enable safer and more effective removal of visceral fat deposits that are having an adverse effect on patients suffering from obesity and metabolic syndrome.

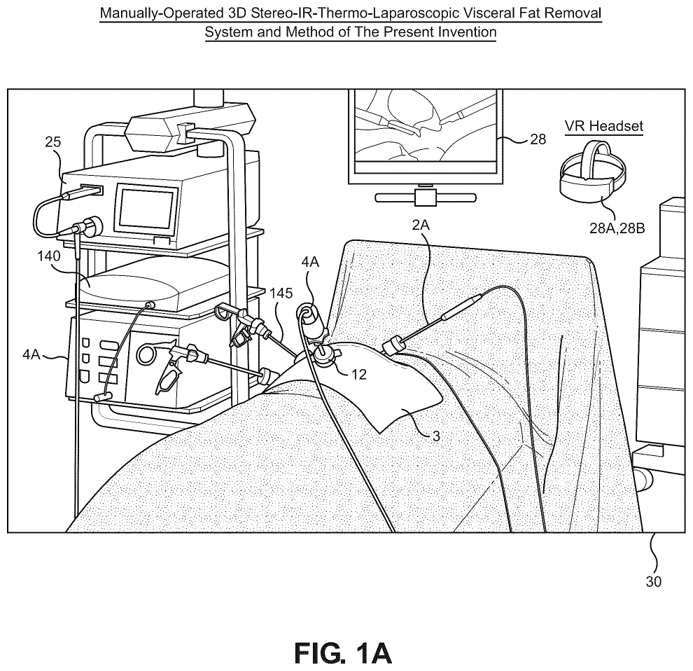

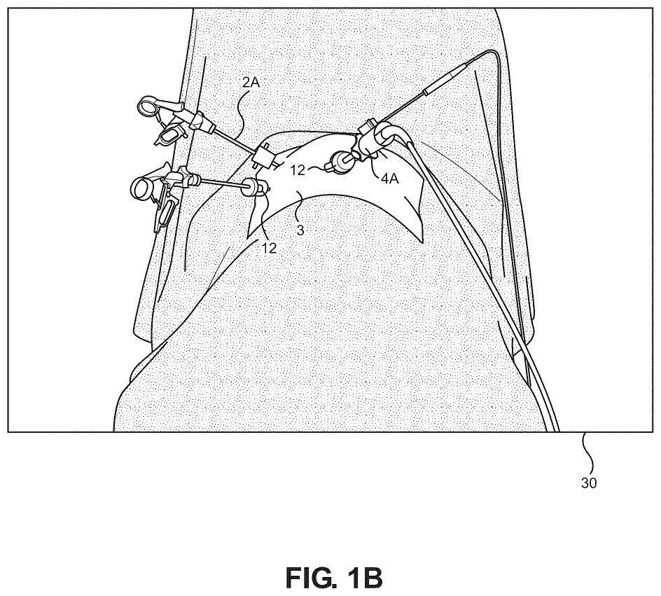

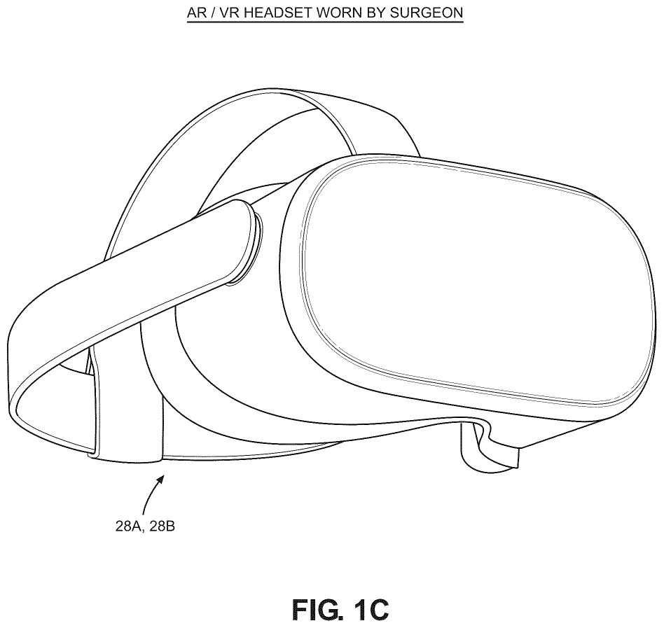

As shown in FIGS. 1A and 1B, a laparoscopic-based visceral fat tissue aspiration system 1 is provided for treating metabolic syndrome in a human patient by non-invasively and safely removing visceral fat tissue deposits from within a human patient having an abdominal region and a mesenteric region within the body of the human patient to ameliorate the metabolic syndrome. The system 1 may be installed in an operating room environment 30, 30′, or elsewhere that meets the necessary surgical operating requirements set by legal code. In general, the human patient 3 will typically have three or more risk factors associated with metabolic syndrome selected from the group consisting of (i) elevated waist-to-hips ratio (Abdominal obesity); (ii) elevated triglycerides, (iii) elevated HDL cholesterol, (iv) hypertension (high Blood Pressure), and (iv) elevated fasting blood sugar (FBS).

As shown in FIGS. 1A and 1B, the 3D-stereoscopic/IR-thermographic laparoscopic-based visceral fat tissue aspiration system 1 comprises: a set of trocars 12 for creating laparoscopy portals through small incisions formed in a human patient's body 3; a source of inert gas 115 for infusion into the abdominal region of the patient so as to cause tenting of the abdominal region and abdominal distension in a human suffering from obesity and likely to benefit from visceral fat removal within the body of the human patient; a 3D-stereoscopic/IR-thermographic laparoscope 2A for insertion through a first one of the trocars and into the abdominal region of the human patient so that a surgeon can capture 3D-stereographic and IR-thermographic video images of the abdominal region of the patient, and display on monitors 28 the captured 3D stereoscopic/IR-thermographic video images within the surgical field of view of the surgeon wearing an necessary 3D-stereo decoding eyewear 120 (e.g. polarized eyeglasses when using polarization-encoding of left and right perspective images); a powered tissue aspiration instrument 4A for insertion through a second one of the trocars 12 and into the mesenteric region of the human patient, wherein the tissue aspiration instrument has an instrument housing and a cannula assembly mounted stationary with respect to the instrument housing; and gripping tools for insertion through a third and optionally fourth trocars 12 installed in the patient's abdomen, for gripping anatomical structures in the mesenteric region during visceral fat tissue aspiration operations. As shown, the 3D-stereoscopic/IR-thermographic laparoscope 4 is adapted for capturing high-resolution digital video images of the mesenteric region of the human patient during visceral fat tissue aspiration operations, and displaying 3D-stereoscopic/IR-thermographic video images, with augmented reality (AR) displaying of cytokine concentration information as a graphical overview on the surgical field of view, for the purpose of providing laparoscopic guidance to the surgeon while safely aspirating visceral fat tissue from the mesenteric region of the human patient to reduce three or more risk factors associated with metabolic syndrome.

As illustrated in FIGS. 1A and 1B, the laparoscopic-based procedure involves a surgical team making one or more limited access portals into the patient's abdomen and using an IR-thermographic/3D-stereographic laparoscopic camera 2A inserted within the abdomen during surgery, and/or a 3D stereoscopic camera monitor 28 for visual assistance to their human vision, while performing the visceral fat aspiration procedure/method in a minimally-invasive fashion, AR-guidance according to the principles of the present invention. Using the 3D-stereoscopic/IR-thermographic laparoscopic-based method, visceral fat can be safely removed from the mesenteric region of a patient to help ameliorate the metabolic syndrome, abdominal obesity and/or type II diabetes of patients.

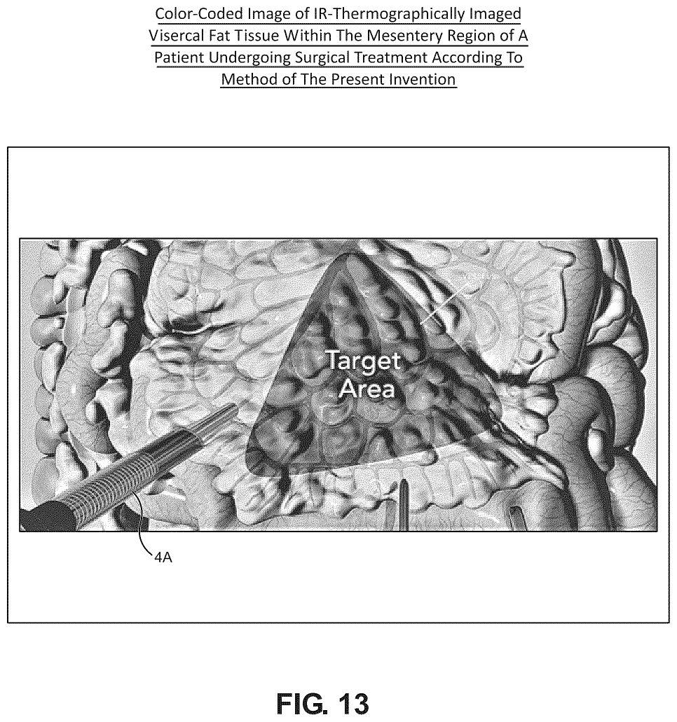

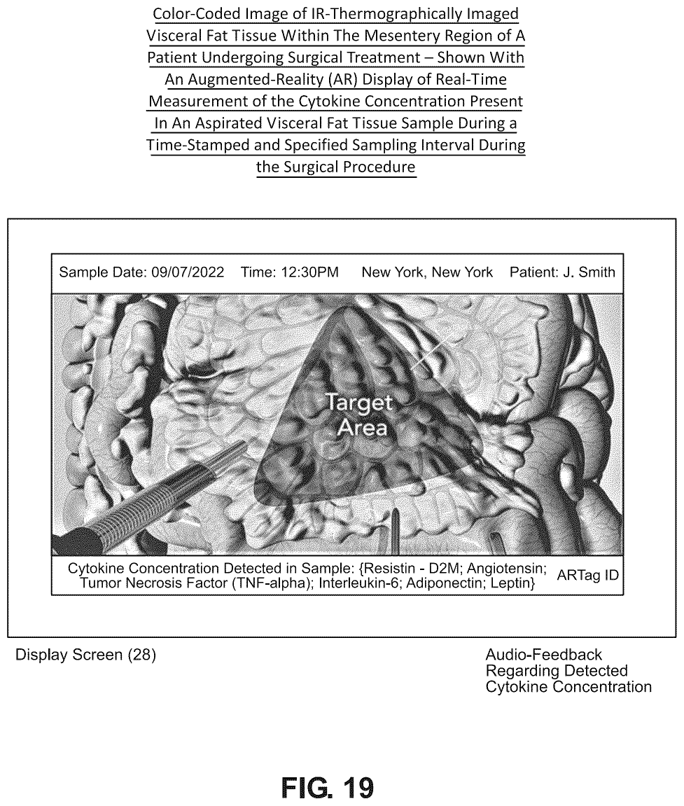

FIGS. 1A and 1B shows a surgical operating room designed and constructed in accordance the principles of the present invention supporting safe non-invasive removal of visceral fat tissue from a patient's mesentery region on an out-patient basis. As shown, the surgical operating room 30 uses (i) manually-operated 3D-stereoscopic (stereo) infra-red (IR) thermographic hand-held laparoscopic instruments 2A supporting (ii) methods of real-time cytokine-sensing and profiling of aspirated fat tissue while the surgeon employs (iii) augmented-reality (AR) guidance and techniques for visual sample-tagging of fat tissue regions being targeted for aspirated removal from the surgical field within the patient's abdominal region. At the same time, the color-coded 3D-stereoscopic/IR-thermographic images are being displayed within the surgeon's stereoscopic field of view (FOV) supported by the 3D-stereoscopic/IR-thermographic laparoscopic instruments of the present invention, as illustrated in FIGS. 13 and 19.

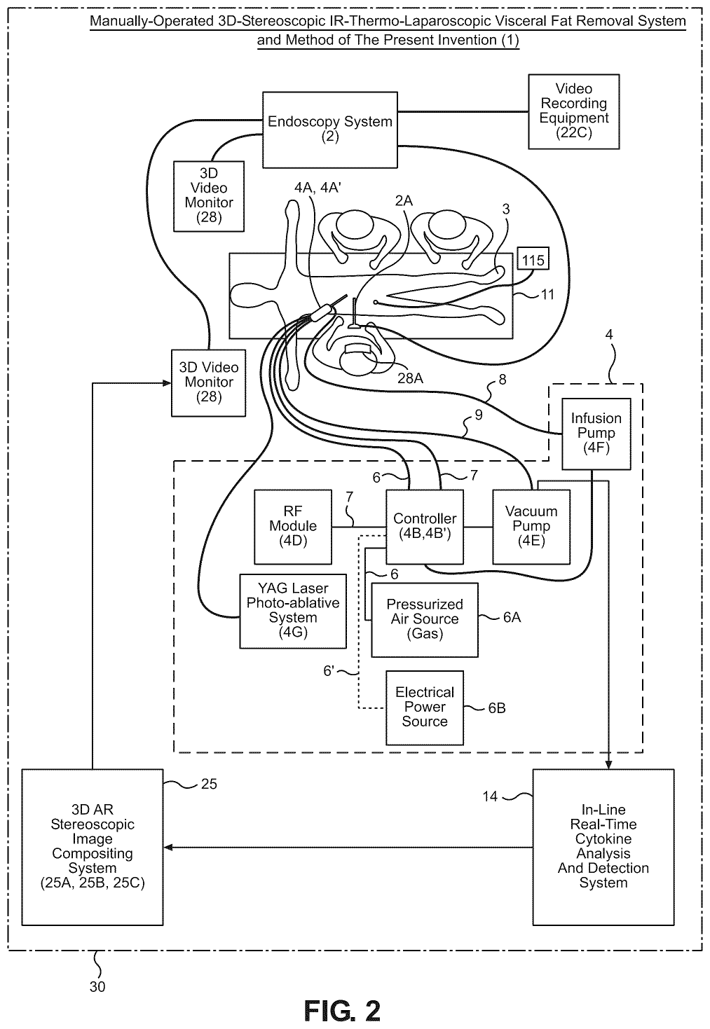

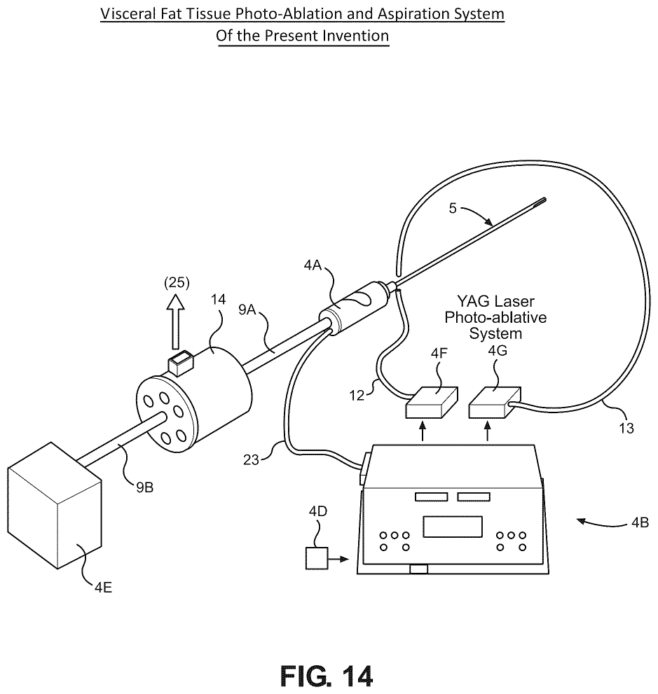

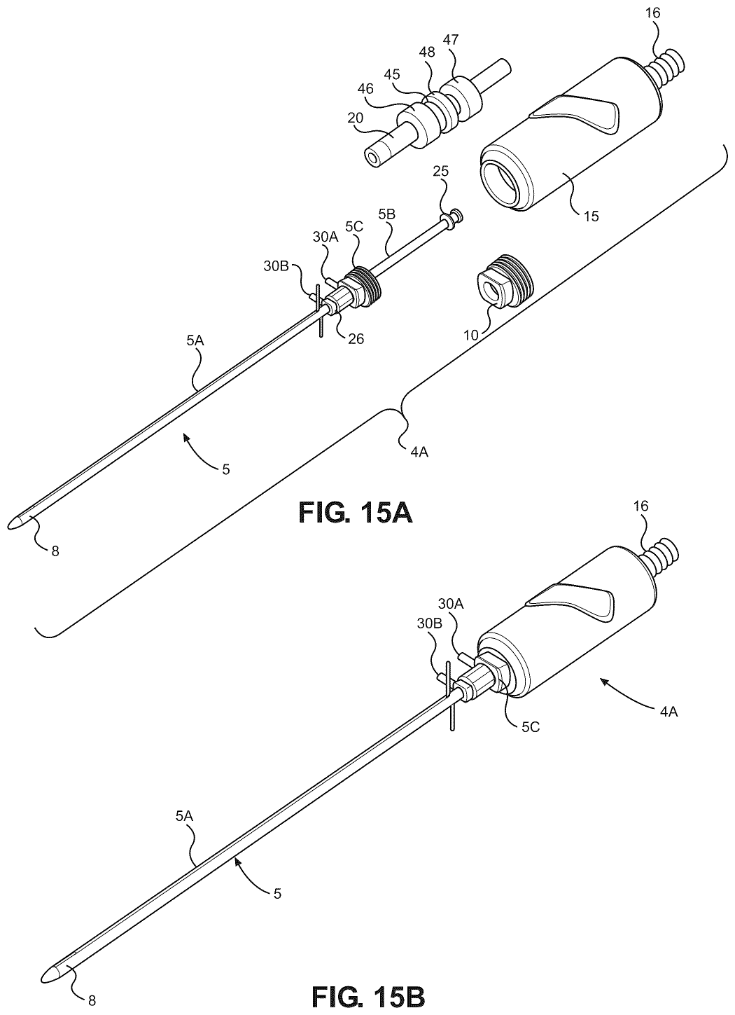

As shown in FIGS. 2, the surgical operating room of FIGS. 1A and 1B, comprises: manually-operated operated 3D-stereoscopic (stereo) infra-red (IR) thermographic laparoscopically-guided power-assisted visceral fat aspiration system 4, adapted for surgically treating an obese patient in the operating room while undergoing a mesenteric visceral fat aspiration procedure. As shown in FIGS. 14, 15A, 15B and 15C, the 3D-stereoscopic (sterco) infra-red (IR) thermographic laparoscopically-guided visceral fat aspiration system 4 includes: (i) a hand-supportable fat aspiration instrument 4A having a hand-supportable housing with a stationary tubing connector 16 provided at the rear of the housing 15 and receiving a length of flexible tubing 9 (9A, 9B) connected to a vacuum source 4E and connecting with cylindrical cannula base portion guide tube 20; a twin tumescent-type cannula assembly 5 having an inner cannula coupled to a cannula drive mechanism disposed within the hand-supportable housing; a real-time cytokine analysis and detection systems 14 installed between the vacuum source 4E and the hand-held aspiration instrument 4A; a 3D AR-stereoscopic image compositing system 2: one or more video display monitors 28; a YAG-laser based photo-ablative system 4G operably connected to the distal end of the tissue aspiration instrument 4A and connected to the YAG laser source via a fiber optical fiber 13.

As shown in FIG. 15C, the cannula drive mechanism 20 is powered by either a source of pressurized air or other gas 6A, 6B, or one or more electro-magnetic coils (i.e. motors) 48, while its stationary outer cannula 5B is releasably connected to the front portion of the hand-supportable housing, as disclosed in U.S. Pat. No. 11,259,862, incorporated herein by reference.

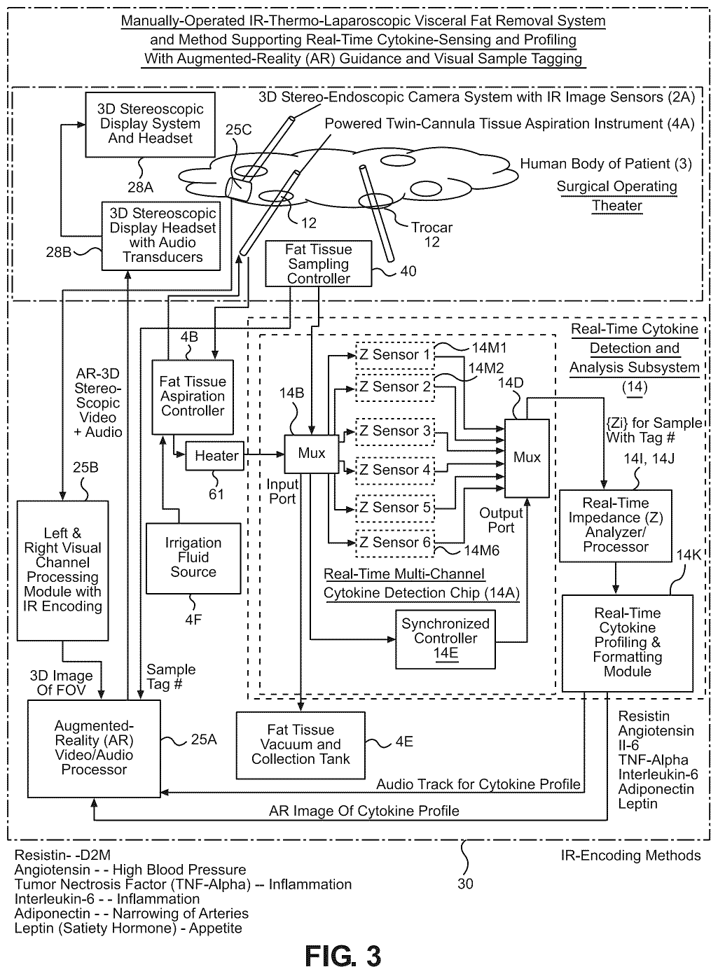

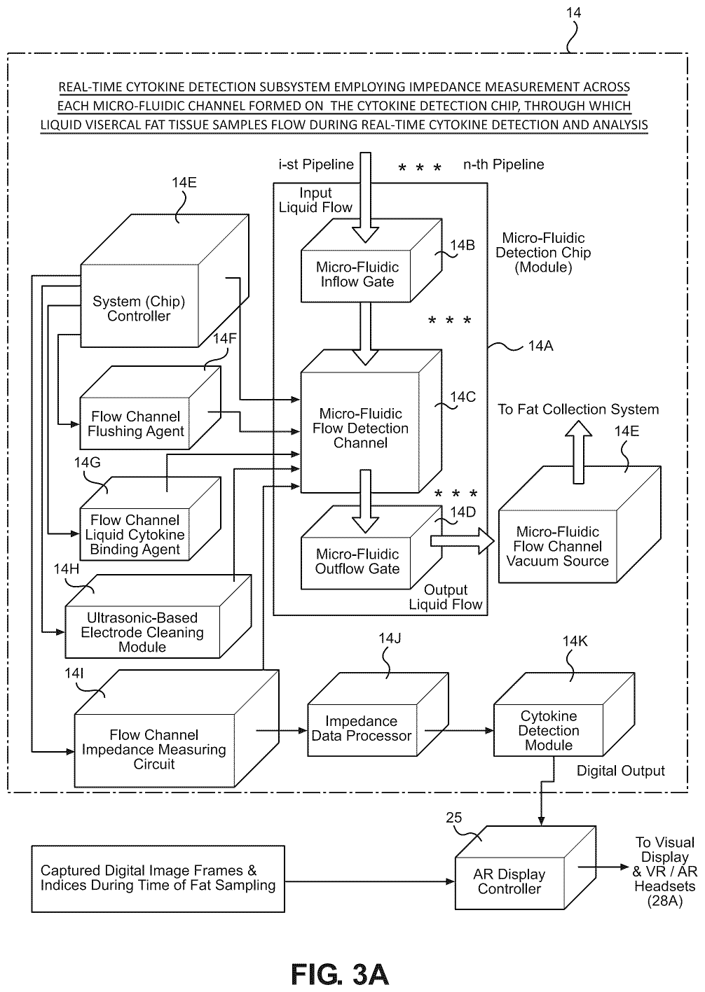

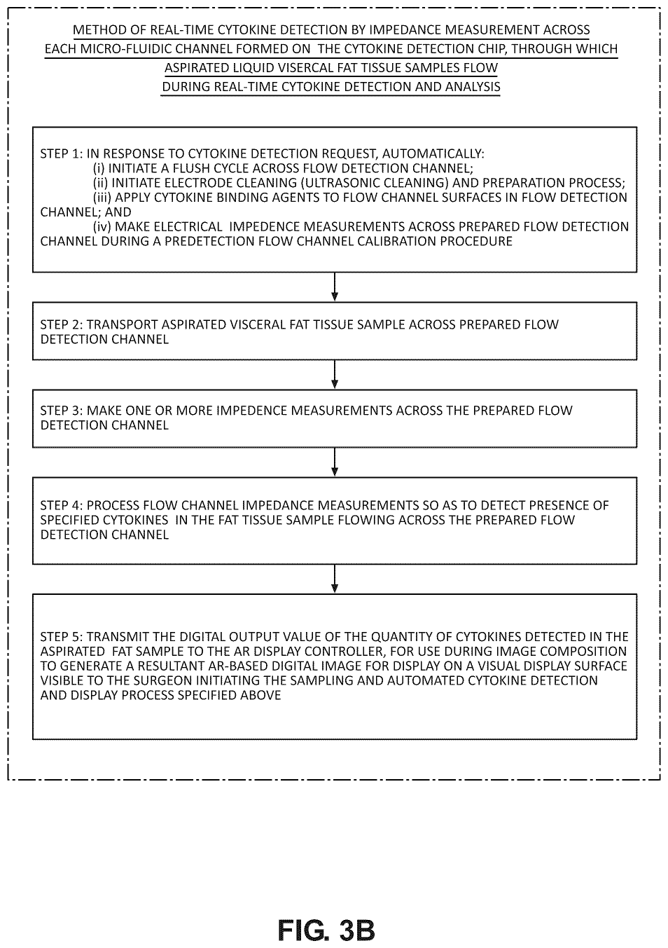

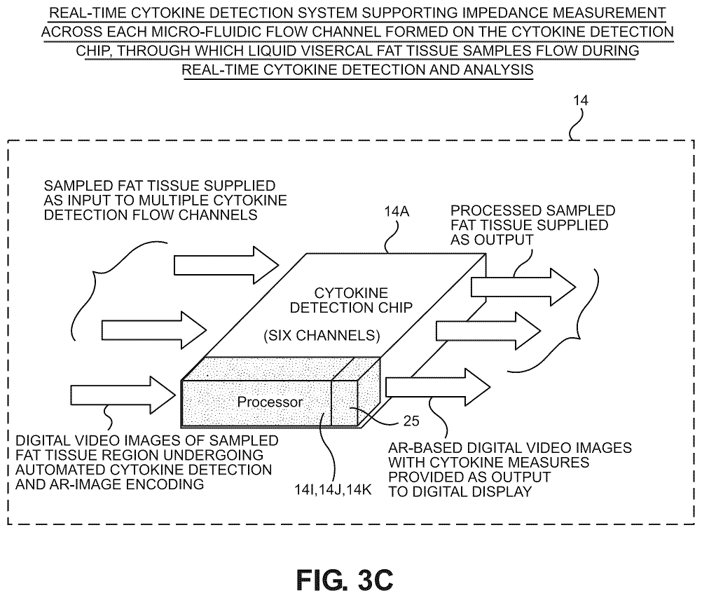

As shown in greater detail in FIGS. 2 and 3, the 3D-stereoscopic (stereo) infra-red (IR) thermographic laparoscopically-guided visceral fat aspiration system 4 further comprises: a system controller 4B for controlling the electro-cautery, irrigation and generation and delivery functions of the YAG photo-ablative laser system 4G supported by the fat aspiration instrument; a 3D stereoscopic/IR-thermographic endoscopy system 2, with video monitors 28 and video recording equipment 22C functioning within the 3D AR stereoscopic image compositing system 2; and the real-time cytokine analysis and profiling system 14 for analysis of aspirated fat tissue and tagging while the surgeon is viewing and aspirating visceral fat tissue graphically represented in 3D-stereoscopic and IR-thermographic images being captured and displayed using the 3D-steregraphic/IR-thermographic-imaging system 2 integrated with 3D stereoscopic laparoscopic tissue aspiration system 4 of the present invention, that is color-coded to the surface temperature of the aspirated visceral fat tissue and surrounding body organs.

As shown in FIG. 3, the 3D-stereoscopic/IR-thermographic endoscopy system 2 employs: (i) a 3D stereoscopic endoscopic digital camera system 2A with left (L) and right (R) image detectors (e.g. CMOS chips) 27A and 27B provided with image formation lenses in front of the image detectors along the left and right visual channels, and IR image sensor(s) 26 mounted or otherwise provided along left, right and/or both left (L) and right (R) visual channels; (ii) a powered twin-cannula tissue aspiration instrument system 4 with reciprocating inner and outer cannulas 5B and 5A; and (iii) a cytokine detection and analysis subsystem 14 for real-time automated detection and analysis of specific kinds and concentrations of cytokine molecular (protein) structures present in aspirated fat tissue samples, and profiling such aspirated samples from specific regions of the abdomen, for collection and indexing, using the augmented-reality (AR) guidance and visual sample tagging methods of the present invention disclosed herein.

The 3D-stereoscopic/IR-thermographic endoscopy system of the present invention 2 employs infrared imaging as a tool for localizing anatomic structures and assessing fat tissue during laparoscopic fat aspiration (and other surgical) procedures. The IR digital camera system 2A incorporated into the two-channel visible laparoscope, is sensitive to thermal energy emitted in the midinfrared range (3-5 microns). Aspiration of cytokine-rich visceral fat regions within the patient's abdomen are aspirated with the aid of the infrared (IR) imaging system 25D working in conjunction with the 3D-stereoscopic imaging system 25C integrated with the 3D-stereoscopic (stereo) infra-red (IR) thermographic based and laparoscopically-guided visceral fat aspiration system 1. Surgeons can easily localize and differentiate structures before tissue aspiration using the visible imaging system, and then using the infrared system during the fat tissue aspiration operations. Assessment of mesentery region of a patient can be conducted using each imaging system.

In the preferred embodiment, the thermal sensitivity of the infrared (IR) imaging system supported by the instrument of the present invention will be sufficient to be highly useful in differentiating between blood vessels and other anatomic structures within the mesentery region of the patient during treatment. Differentiation of the blood vessels and arteries and fat tissue localization is visually enabled using the infrared (IR) camera system 26 having milli-kelvin (mK) thermal sensitivity in the range of >50 mK and >35 mK. Also, the IR imaging subsystem 26 permits assessment of bowel perfusion during laparoscopic fat tissue aspiration around and about mesenteric vessels. Infrared imaging of this region during surgical operations will improve differentiation and localization of anatomic structures, and also allow assessment of physiologic parameters such as perfusion not previously attainable with visible laparoscopic techniques, and for this reason, should serve as a powerful adjunct to laparoscopic surgery.

As shown in FIG. 3, the real-time cytokine detection and analysis subsystem 14 is operably connected to the aspiration instrument 4A which is operably connected to the fat tissue aspiration controller 4B. As shown, the cytokine detection and analysis subsystem 14 comprises: a real-time multi-channel cytokine detection chip 14A having an input multiplexer 14B with an input connected to the fat aspiration instrument 4A, and an output supplying a plurality of cytokine detection channels 14M1 through 14M6. Each detection channel supporting a cytokine sensor supplies an output data signal from the cytokine detection chip 14A that is representative of the impedance presented by the aspirated visceral fat tissue in the detection channel of the chip, and indicative of the kind/type and quantity of cytokine chemical molecules present in the specific detection channel, (i.e. Resistin—involved in D2M; Angiotensin—involved in high blood pressure; Tumor Necrosis Factor (TNF-alpha)—involved in inflammation; Interleukin-6—involved in inflammation; Adiponectin—involved in narrowing of arteries; and Leptin (satiety hormone) involved in appetite satiation.

As shown in FIG. 3, sensor outputs from the cytokine detection chip 14A are provided to an output multiplexer 14D which is controlled by a synchronized controller 14E. The controller 14E controls the transfer of data signals from each sensor channel 14M1 through 14M6 into the multiplexer 14D, one channel processing cycle at a time, for subsequent processing and analysis in the real-time impedance analyzer/processor 14I, 14J (e.g. programmed microprocessor realized a system on a chip SOC architecture). Further technical details on the design and construction of each cytokine defection channel, for real-time rapid-sample-to-answer, label-free detection of cytokines and other biomarkers (i.e. Resistin—D2M; Angiotensin—high blood pressure, Tumor Necrosis Factor (TNF-alpha)—inflammation, Interleukin-6—inflammation, Adiponectin—narrowing of arteries, and Leptin—satiety hormone) can be generally found in the semiconductor electronic-chemical detection chip art. Of notable interest is the 2021 technical publication titled “A Microchannel-Based Impedance Sensor On An Insertable Microneedle For Real-Time In Vivo Cytokine Detection” by Naixin Song, et al published in Microsystems & Nanoengineering (2021), Vol. 7, No. 96, incorporated herein by reference.

In addition a printed circuit board can be designed to act as a biosensor for a specific molecule. The bioreporter is operably linked to the working electrode cable.

When a surgeon is aspirating selected regions of the mesentery and other abdominal regions during the practice of obesity treatment, the microfluidic-cell based cytokine detection chip 14A automatically analyzes and profiles, in real-time, visceral fat samples flowing from the aspiration instrument 4A into the microfluidic-cell based channels of the cytokine detection and analysis chip 14A, while visually indicating on AR-based GUI display screens 28, shown in FIGS. 13 and 19, the current metabolic activity of selected samples of aspirated fat tissue, measured and quantified in terms of the following cytokines and their respective concentrations, namely, for example: Resistin— contributing to D2M; Angiotensin—contributing to high blood pressure; Tumor Necrosis Factor (TNF-alpha)—contributing to inflammation; Interleukin-6—contributing to inflammation; Adiponectin—contributing to narrowing of arteries; and Leptin— (satiety hormone)— contributing to appetite control.

In the preferred embodiment, microfabrication technology will be used to create a microfabricated electrical impedance-based sensing platform (i.e. sensor chip) 14A for label-free, in situ detection of cytokines present in continuously aspirated visceral fat tissue sampled and collected from a patient in liquified form, using the apparatus schematically illustrated in FIGS. 2 through 8.

As illustrated in FIG. 3A, the real-time cytokine sensor chip 14A relies on a plurality of antibody-functionalized microchannels, resulting in high selectivity, a wide detection range, and picomolar sensitivity. Such impedance-based cytokine sensors (e.g. in the form of microchannels support micro-fluidic flow of aspirated fat tissue from an input port on the chip package, to an output port on the chip package) are formed on glass or silica substrates, and are able to detect low concentrations of cytokines moving through the real-time cytokine detection device of the present invention.

As show in FIG. 3A, the in-line multi-microchannel (i.e. sensor) 14 is realized as a real-time cytokine detection and analysis system comprising: a number of subcomponents, namely, (i) a micro-fluidic detection chip module 14A, wherein each cytokine sensing channel or pipeline (14M1 . . . 14M6) realized on the chip 14A has an input micro-fluidic inflow gate (i.e. multiplexer) 14B operably connected to the powered aspiration instrument 4A inserted and guided within the patient, and also connected to a micro-fluidic flow detection channel/pipeline (14M1, . . . 14M6) that is connected to an output microfluidic outflow gate (i.e. multiplexer) 14D, that is operably connected to a flow channel vacuum source 4E, (ii) a flow channel flushing agent module 14F in fluid communication with each micro-fluidic flow detection channel 14C, for flushing the channel with an appropriate flushing agent under the control of the system controller 14E, (iii) a flow channel liquid cytokine binding agent module 14G in fluid communication with each micro-fluidic flow detection channel 14C, for supplying the channel with an appropriate cytokine binding agent 14G under the control of the system controller 14E, suited for the particular cytokine to be detected within this specific flow channel 14C on the chip 14A, (iv) a ultrasonic-based electrode cleaning module in fluid communication with each micro-fluidic flow detection channel 14C, for ultrasonically cleaning the channel with ultrasonic energy and applied fluid under the control of the system controller 14E; (v) a flow channel impedance measuring module 14I in fluid communication with each micro-fluidic flow detection channel 14C, for electrically measuring the impedance (Z) characteristics of the fat sample flowing within the sensing channel under the control of the system controller, (vi) an impedance data processing module 14J in communication with the flow channel impedance measuring circuit module 14I for processing the electrical data collected by the impedance measuring circuit 14I under the control of the system controller 14E, (vii) a cytokine detection module 14K in communication with the impedance data processor 14J for processing the data and detecting the type and quantity (i.e. concentration) of cytokine molecules in the specific flow sensing channel aboard the chip device 14A, for providing digital output to the AR display controller 25 while it is receiving captured digital image frames and indices during the time of fat sampling, and compositing the data input to generate a composite video output for visual display on the 3D stereoscopic AR display system 2.

In the preferred embodiment, the cytokine detection module is microfabricated as a microchannel impedance-based protein-detection sensor chip designed and constructed for rapid-sample-to-answer, label-free detection of cytokines, other biomarkers, and proteins of interest, supporting real-time in vivo quantification and detection of cytokines and other biomarkers is achieved by continuous measurement of the electrical impedance changes between the (gold-plated) sensor electrode surfaces inside the microchannel, arising from the specific binding of the target protein to the cytokine antibody already present/deposited within the microchannels, during the set-up process. Further, specific binding of the target protein (i.e. cytokine) to the cytokine antibody on the microchannel surface affects ion transport inside the microchannels, resulting in a change in impedance between the two electrodes, in the cytokine-detection microchannel, thereby enabling real-time monitoring of both the antibody attachment process and target protein binding, by continuously capturing the change in electrical impedance across the microchannel.

FIG. 3B describes a method of real-time cytokine detection by impedance measurement across each micro-fluidic channel formed on a cytokine detection chip 14A, through which liquid visceral fat tissue samples flow during real-time cytokine detection and analysis carried out as part of a non-invasive method of visceral-fat removal from patients suffering from metabolic syndrome.

In general, the system (chip) controller 14E, implemented using a programmed microprocessor, FPGA or other digital processing device, opens and closes the inflow and outflow gates 184 and 14D to clear out the microflow channels 14C in a periodic manner as illustrated in FIGS. 2A and 2B. Once the microchannels 14C have been cleaned and then precoated with their respective cytokine antibody material, and are ready to collect and bind to respective cytokines in the next fresh sample of tagged/indexed visceral fat tissue, the system controller 14E closes the outflow gates 14D and opens the inflow gates 14B to allow the new preheated sample of liquified visceral fat tissue sample to flow into, and fill up, the array of microchannels 14C, and then the inflow gates are closed for the electrical impedance measurement operations described in FIG. 3B. The details of this sequential process outlined in FIG. 3B, are automatically repeated over and over again in a cyclical manner during the course of tissue aspiration operations using the system 4, and the detected cytokine protein content and concentration data within aspirated tissue samples are provided to the AR display processor 25 for automated image compositing and display using AR display methods described herein.

As shown in FIG. 1C, this includes the surgeon wearing conventional AR/VR headgear 28A, such a Meta Quest 2 Advanced All-in-One VR Headset (28), for example, to view 3D stereoscopic and IR-thermographic images with AR-encoded cytokine content displayed in a heads-up fashion, as shown in FIG. 19. However, it is understood that any VR headset can be used to insert the surgeon into a virtual world, or metaverse, and interact with his or her surgical workspace (i.e. field of view) while aspirating and sampling visceral fat tissue and viewing AR-display images with IR thermographic color-encoding and cytokine-concentration information displays provided in a heads-up manner, as shown in FIG. 19, for the benefit and advantage of the surgeon during the practice of the tissue aspiration operation according the to present invention.

As shown in FIG. 3B, the Step 1 in the method carried out in the system of FIG. 3A involves: in response to a cytokine detection request, (i) automatically initiating a flush cycle across a flow channel 14C, (ii) initiate electrode cleaning (i.e. ultrasonic cleaning) across the sensing flow channel, (iii) applying cytokine binding agents to the flow channel surfaces, and (iv) making electrical impedance measurements across the prepared flow channel during a pre-detection flow channel calibration procedure. The flush or rinse cycle indicated above may involve the use of ethanol, isopropyl alcohol, fenofibrate or heated saline which may be pulsed/oscillated to clear and clean the microchannels between sampling sessions.

As indicated in Step 2, the method involves transporting aspirated visceral fat tissue sample across a prepared flow channel.

As indicated in Step 3, the method involves making one or more electrical measurements across the prepared flow channel.

As indicated in Step 4, the method involves process flow channel impedance measurements so as to detect presence of specified cytokines in the fat tissue sample flowing across the prepared flow detection specified.

As indicated in Step 5, the method involves transmitting the digital output value of the quantity of cytokines detected in the aspirated fat sample to the AR display controller, for use during image composition to aspirated fat sample to the AR display controller 25 for use during image composition to generate a resultant AR-based digital image for display on a visual display surface visible to the surgeon initiating the sampling and automated cytokine detection and display process specified above.

FIG. 3C illustrates the real-time cytokine detection system 14 supporting impedance measurement across each micro-fluidic flow channel formed on the cytokine detection chip 14A. Through each micro-fluidic flow channel 14C, liquid visceral fat tissue samples flow during real-time cytokine detection and analysis for processing in the micro-fluidic flow channels and output from the chip 14A. As shown in FIG. 3C, the digital video images of a sampled fat tissue region undergo automated cytokine detection and AR-image encoding, wherein digital video images are provided to the chip for input processing, and as output, the chip automatically generates AR-based digital video images provided with cytokine measures for digital display.

In the preferred embodiment, each cytokine-sensing microchannel (i.e. bio-chemical sensor) 14C formed on the sensor chip 14A consists of a ˜20 μm wide microchannel, laser micromachined into fused silica plate, and provided with electrodes that are photolithographically configured on the plate. Specifically, at each cytokine-sensing microchannel, a pair of gold sensor electrodes are separated by a 40 nm insulating layer of aluminum oxide. The basic principle of the electrical-impedance sensor is continuous measurement of the electrical impedance change between (i) the sensor electrodes arising from the specific binding of the target protein (i.e. cytokine) to the cytokine antibody applied to and present within the micro-sensing channel prior to cytokine sensing operations carried out within the in-line device 14. During the operations specified in FIG. 3B, the cytokine antibody is applied to the gold electrode surface, inside the microchannels. Specific binding of the target protein (cytokine) to this antibody coating affects ion transport inside the microchannel, resulting in a change in impedance between the two electrodes. As a result, real-time monitoring of both the antibody attachment process and target protein binding, can be achieved by continuously capturing, recording and processing this change in electrical impedance.

Suitable antibody coatings that can be used to promote and produce microchannel binding surfaces for accumulation or buildup of protein molecules (i.e. for each cytokine type to be detected via electrical impedance, spectral and/or other measurement principles) will be selected based on the particular cytokine molecules to be automatically detected and analyzed by the system. For example, recombinant anti-resistin antibody [EPR2334-171] [AB275878] coating may be used to bind to Resistin; monoclonal anti-angiotensinogen antibody[BGN/KA/4H) coating may be used to bind to Angiotensin; rabbit monoclonal anti-TNF alpha antibody [EPR19147] may be used to bind to Tumor Necrosis Factor (TNF-alpha); rabbit monoclonal anti-IL 6 antibody [EPR21710] coating may be used to bind to Interleukin-6; rabbit monoclonal anti-adiponectin antibody [MFCDO3454695] coating may be used to bind to Adiponectin; and rabbit monoclonal anti-leptin [ab16227] antibody coating be used to bind to Leptin. Other suitable antibody coatings will occur to those skilled in the art without undue experimentation, for the cytokines indicated, and others that will be useful to detect and analyze in any given application.

In the preferred embodiment, each microchannel sensor (i.e. sensing channel) 14C realized on the cytokine detection chip 14A can be prepared on a thin fused silica substrate having, for example, 100 mm diameter, 500-micron thickness. Vapor deposition of conductive and insulative layers is carried out, and then the microchannels are photolithographic etched in each sensor channel using photo-lithographic etching techniques well known in the art. The bonding pads are then formed on the chip device. More specifically, a first thin adhesion layer (e.g. chromium of 5 nm thickness) followed by a gold layer (e.g. 110 nm thickness) can be deposited on the silica substrate, by utilizing physical vapor deposition and defined by using photolithography and liftoff, yielding the lower electrode, interconnecting line, and bonding pad. A first 40 nm aluminum oxide (Al₂O₃) insulation layer may be deposited by using atomic layer deposition. A second 5 nm adhesion layer of chromium followed by a 100 nm gold electrode can be positioned on top of the first electrode in the sensing region, as well as a separate interconnecting line and bonding pad. A second 40 nm Al₂O₃ layer can be deposited to insulate the sensing platform. A laser lithography system can be utilized to pattern microchannels (2 μm diameter) in the electrode overlapping region. The microchannels can be prepared with sequential reactive ion etching of two layers of Al₂O₃ and wet etching of gold until the bottom gold electrode is exposed. Gold bonding pads can be lithographically defined and exposed for connection and recording. Each sensor channel (14C) is then micromachined into a suitable shape using an excimer laser micromachining system. The two electrodes have an overlapping region (20 Å˜20 μm) in dimension, on which an array of at least 6 individual 2 μm diameter microchannels (14C) are configured on a single chip 14A. External electrical connections to the gold bonding pads can be provided by the use of commercially available connectors.

In general, the magnitude and phase behavior of the electrochemical impedance of each sensor channel 14C will vary as a function of frequency for a typical sensor when immersed in and/or exposed to various aspirated fat liquid environments expected when practicing the present invention. To correlate electrochemical responses to the electrical properties of the sensor 14C, collected impedance data can be fitted into an equivalent circuit model representing, for example, (i) the outside sensing region and (ii) the microchannel sensing region of each microchannel sensor 14C constructed as described above.

In the outside sensing region, the effects of two thin oxide-insulated gold traces near the sensing region can be modeled as a branch comprising: a series arrangement of capacitors representing the dielectric properties of the protecting Al₂O₃ layers on the lower and upper gold traces, respectively; and a resistor used in the circuit model for quantifying the resistance to the movement of (protein) ions through the sampled visceral fat liquid present in the outside sensing region.

Within the microchannel sensing region, one or the capacitors can be used in the model to reflect the dielectric property of the Al₂O₃ insulation layer in the overlap region of two gold electrodes. A parallel combination of constant phase elements can be used to represents the interfacial capacitance. These interfacial capacitances, together with charge transfer resistances can be used to model the two gold-electrolyte interfaces within the microchannels 14C of the cytokine detection array chip 14A. A first resistance can be employed to quantify the resistance to the movement of ions through the solution inside the microchannels. A capacitor can be used to describe the dielectric property of Al2O3 on the top gold electrode, while the second resistor is used to represent the resistance to the movement of ions through the solution in the region adjacent to the microchannel array extending from the microchannel to the uppermost Al₂O₃ layer. Such electrical property model of micro-electronic devices is generally known in the semiconductor instrument art.

To determine the parametric values of the various circuit components described above, an equivalent circuit model will be fitted to multiple sets of measured impedance characteristic data collected for each sensor channel under design. The fitting of measured impedance data can be performed utilizing tools, instruments and software well known in the instrument design art, including variable frequency signal generators, oscilloscopes and the like. Overall, the extracted parametric values should be reasonably correlated with estimated values calculated from sensor geometry and electrical properties of materials used to construct the cytokine detection sensing chip. A suitable parameter for monitoring fat-dependent impedance changes across a sensor micro-channel, can be determined through modeling and undue laboratory experimentation. Such laboratory experimentation may reveal and confirm that the real part of the sensor microchannel impedance, determined at a specific frequency (e.g. 100 kHz), will be the suitable parameter for monitoring fat-dependent impedance changes across a sensor micro-channel 14C.

In practice, these automatic multi-fluidic channel cytokine sensors 14C will used to carry out real-time in vitro detection and quantification of specified cytokine concentration levels in the aspirated liquified visceral fat samples being collected from obese patients undergoing the endoscopic out-patient procedure of the present invention. To assess this automated in vitro cytokine sensor functionality, cytokine concentration levels in aspirated fat tissue samples, collected from the same patient, will be quantified and calibrated using traditional laboratory test procedures on such samples, to ensure accurate real-time in vitro sensor readings in aspirated fat tissue samples.

While the electrical impedance across each sensor microchannel is continuously measured to detect and quantify the presence of a specific cytokine present in the aspirated visceral fat fluid sample, during the EVL outpatient procedure, on a real-time basis, to help the surgeon remove the most metabolically active visceral fat tissue from the patient, other methods may be used to realize the cytokine detection chip of the present invention using different measurement principles and physics. Examples of techniques include, but are not limited to: using nanotechnology to design printed circuit boards that have terminal connections spatially configured to directly make contact with bondable sites on the cytokine molecules.

### Specification of the Manually-Operated Power-Assisted Fat Aspiration Instrument System of the Present Invention Shown Deployed in a Surgical Operating Room Environment

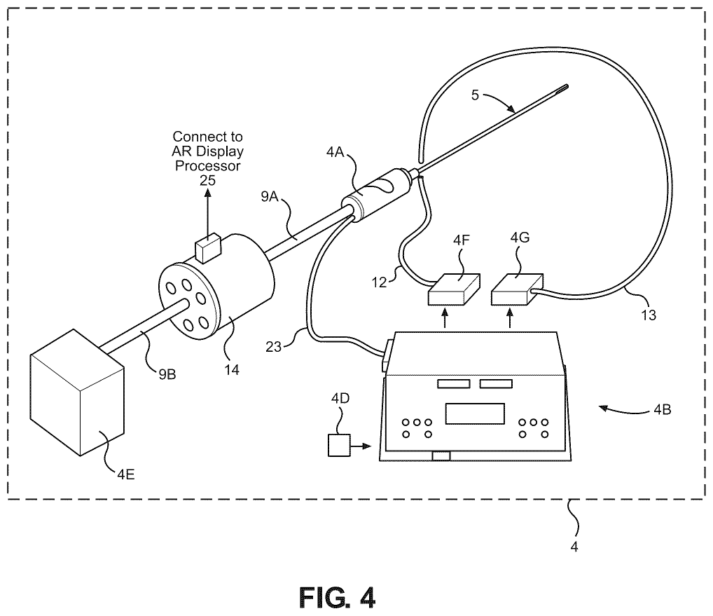

As shown in FIGS. 4, 14A, 14B and 14C, the manually-operated power-assisted fat aspiration instrument system 4 is deployed in the surgical operating room environment of FIGS. 1A, 1B, 2, 3, and 3A-3C. The manually-operated power-assisted fat aspiration instrument 4 has a twin-cannula assembly 5 supporting at least three-functions (i.e. tumescent infusion, electro-cautery, and YAG-based photo-ablative laser illumination and delivery) about the aspiration aperture 8 during visceral fat aspiration operations.

The visceral fat aspiration instrument system 4 comprises a hand-supportable fat aspiration instrument 4A having a hand-supportable housing 15 with a stationary tubing connector 15 provided at the rear of the housing 15 and receiving a length of flexible tubing 9B connected to a vacuum source 4E, and including a twin-cannula assembly 5 coupled to a cannula drive mechanism 48 disposed within the hand-supportable housing 15 and powered by an external power source 6B (e.g. electrical power signals, pressurized air-streams, etc.) so as to periodically exert forces on the cannula base portion along the longitudinal axis of the cannula assembly (i.e. coaxially exerted on the cannula base portion) and cause the hollow inner cannula base portion to reciprocate within the cylindrical (inner cannula base portion) guide tube 20, while tissue is being aspirated along the cannula lumen, through the lumen formed in the cannula base portion, through the cylindrical guide tube 20 and through the stationary tubing connector 16, along the flexible tubing 9B towards the vacuum source 4E.

As shown, the hand-supportable fat aspiration instrument 4 comprises: (i) a hand-supportable housing having: (i) a front portion and a rear portion aligned along a longitudinal axis; (ii) an interior volume and a cylindrical guide tube mounted within the interior volume; (iii) a cannula drive mechanism disposed adjacent the cylindrical guide tube; and (iv) a stationary tubing connector coaxially mounted to the rear portion of the hand-supportable housing along the longitudinal axis, connected to the cylindrical guide tube, and having an exterior connector portion permitting a section of flexible aspiration tubing to be connected at its first end to the exterior connector portion, and where the second end of the section of flexible tubing is connected to a vacuum source.

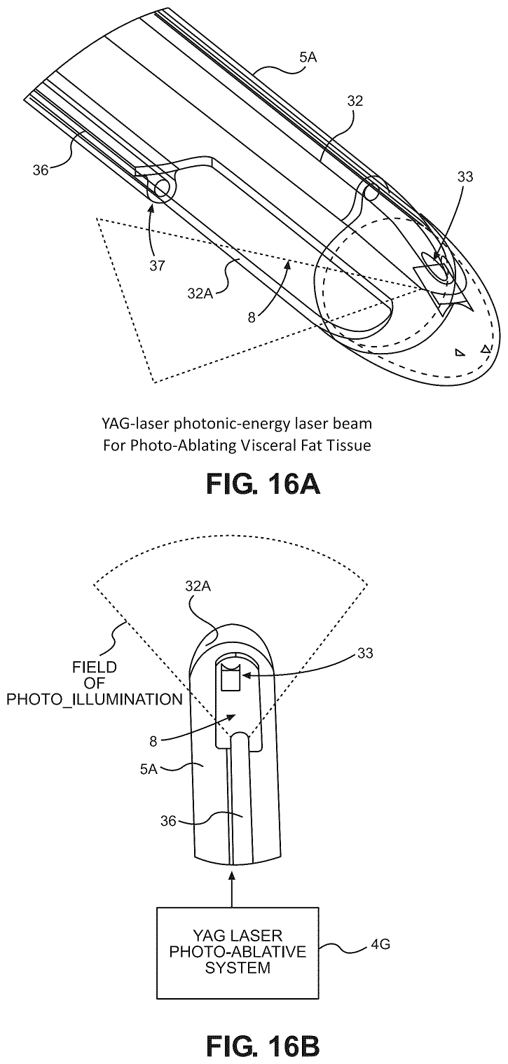

During operation, a stream of irrigation fluid 4F is automatically pumped from the base portion 5C of the outer cannula 5A to the distal portion thereof, along a micro-sized fluid conduit 36 formed along the surface walls of the outer cannula 5A, and released into the interior distal portion of the outer cannula through a small opening 37 formed therein, as shown in FIG. 16A, for infiltration and irrigation of tissue during aspiration in order to facilitate pump action. Selected aspirated fat tissue samples are processed by the real-time cytokine detection and analysis (chip) system 4A for the purposes of the present invention described in detail herein.

### Specification of the 3D-Stereoscopic IR-Thermographic Laparoscopic Surgical Robotic System of the Present Invention

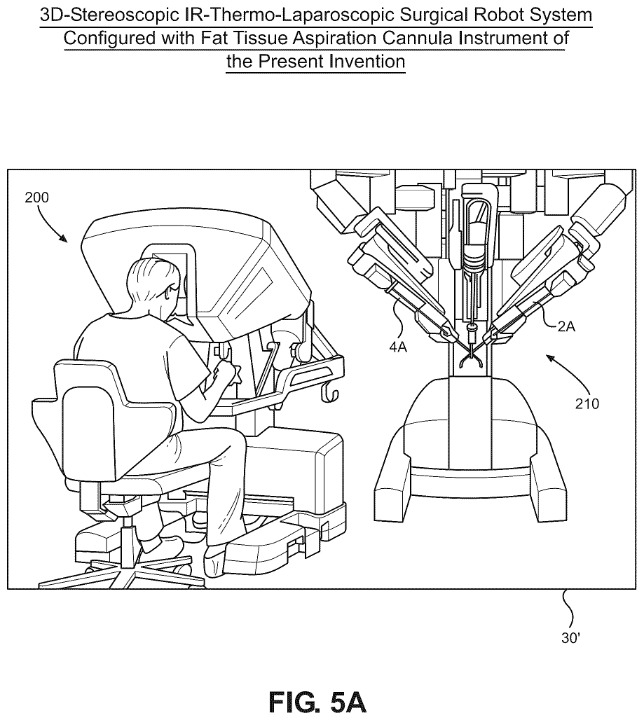

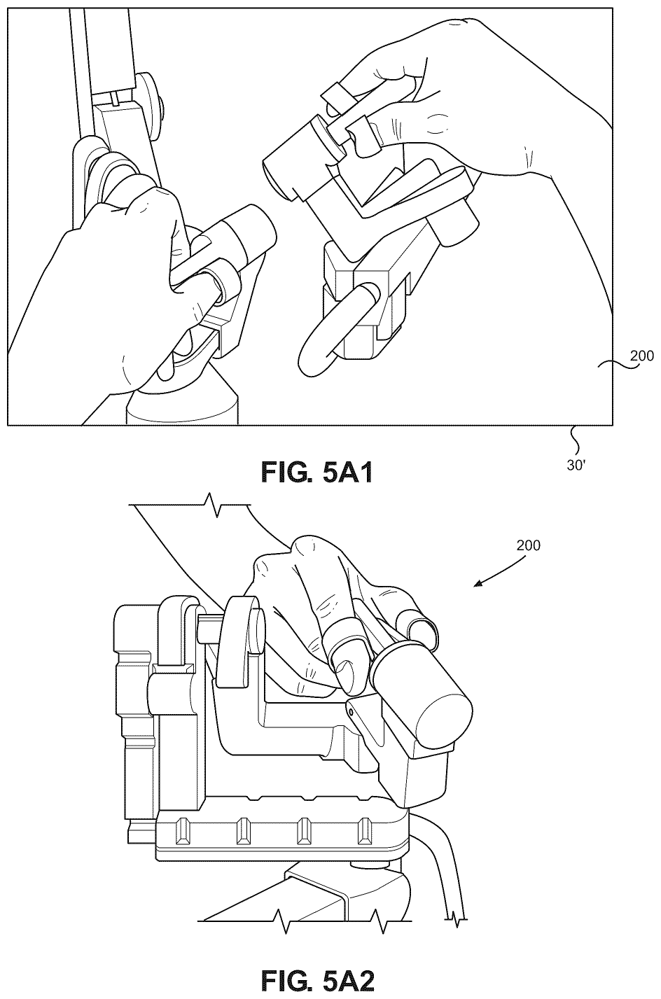

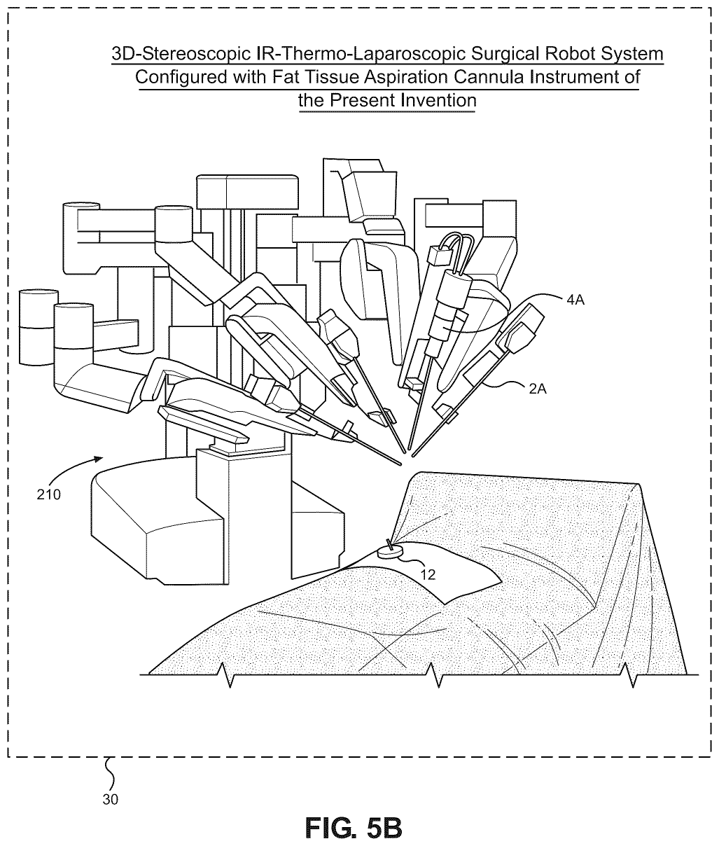

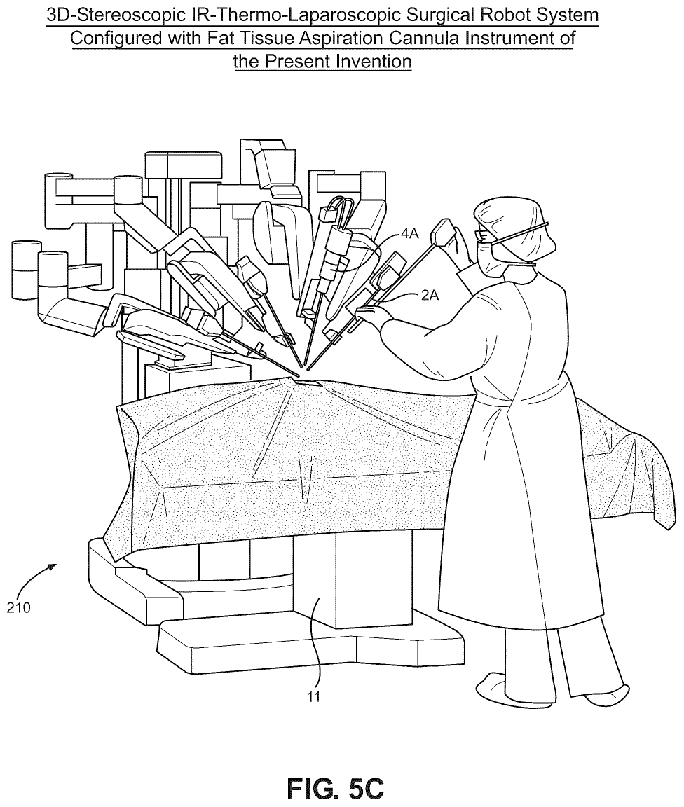

The 3D-Stereoscopic IR-Thermographic Laparoscopic Surgical System described above 1 can be practiced in a robotically-controlled mode of operation, as shown in FIGS. 5A through 11, and described below.

FIG. 5A shows a Robotically-Controlled 3D-Stereoscopic IR-Thermographic Laparoscopic Surgical (Robotic) System 1′ configured with the powered fat tissue aspiration cannula instrument subsystem 4, and the real-time cytokine detection and analysis subsystem 1, handled by a multi-arm surgical robotic system 210. As shown in FIGS. 5A through 5C, the multi-arm surgical robotic system 210 can be realized by a suitably adapted da Vinci® Surgical Robotic System produced and sold by Intuitive Surgical, Inc. of Sunnyvale, California, or using a functional similar system suitable for the application at hand.

FIGS. 5A1 and 5A2 show a hand-controlled surgical interface console subsystem 200 employed in the Robotically-Controlled 3D Stereo-IR-Thermo-Laparoscopic Surgical Robot System 1′ depicted in FIG. 5A.

FIG. 5B shows the Robotically-Controlled 3D Stereo-IR-Thermo-Laparoscopic Surgical Robot System 1′ in FIG. 5A, configured with the powered fat tissue aspiration cannula instrument 4A held and controlled by a robotic navigation subsystem 210 that is programmed to handle the other surgical instruments, such as digital cameras, grippers, etc. depicted in FIGS. 2 and 3.

FIG. 5C shows the Robotically-Controlled 3D Stereo-IR-Thermo-Laparoscopic Surgical Robot System 1′ in FIGS. 5A and 5B, configured with the powered fat tissue aspiration cannula instrument subsystem 4 held and controlled by a robotic subsystem 210 handling the other surgical instruments depicted in FIGS. 2 and 3.

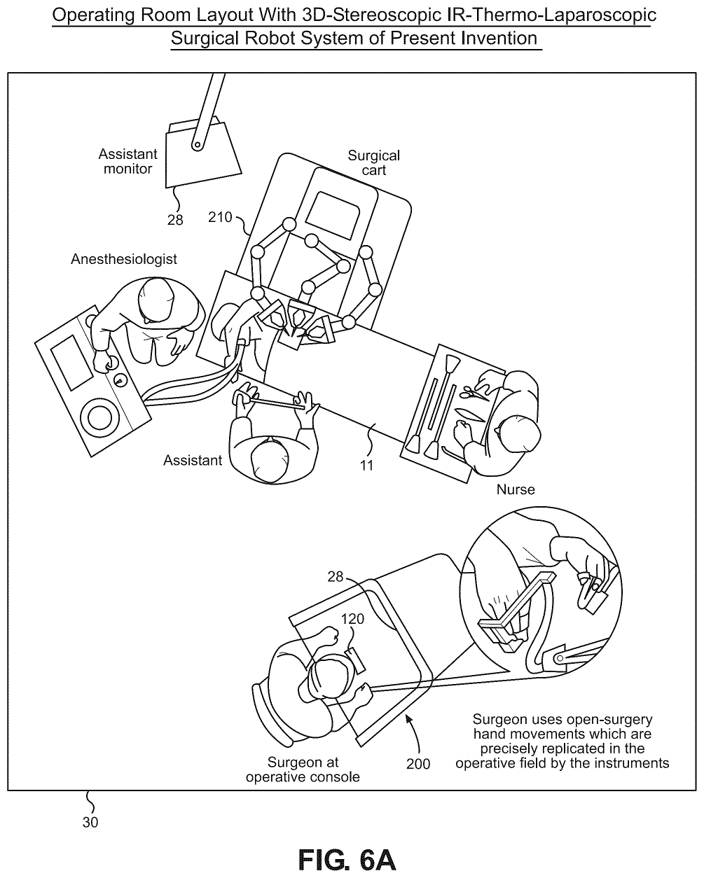

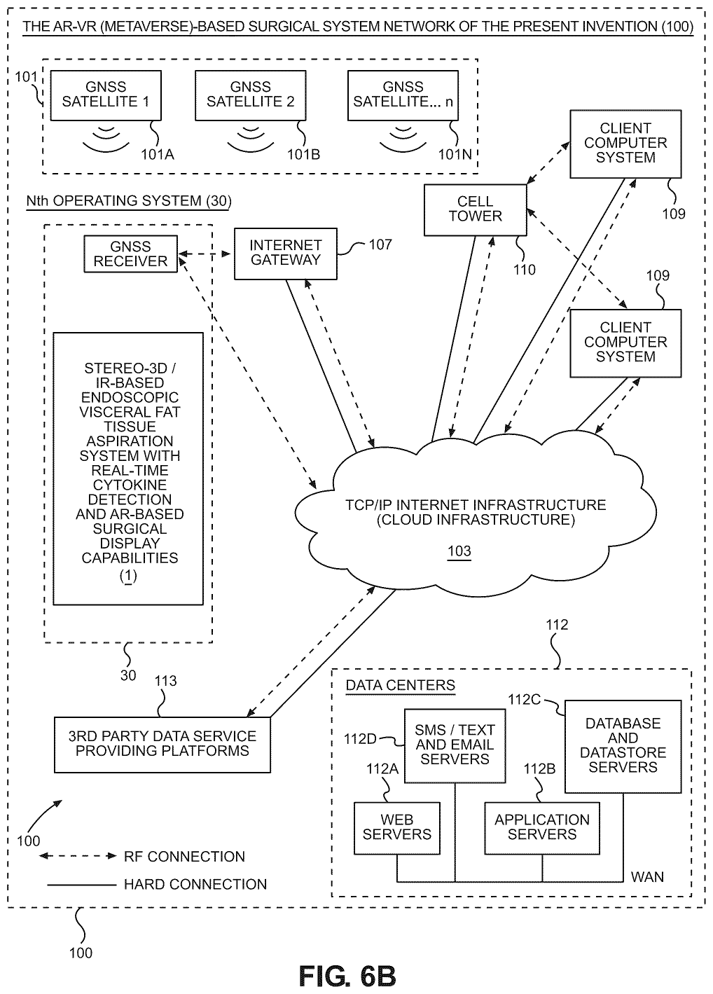

FIG. 6A schematically illustrates the layout of an operating room provided with the Robotically-Controlled 3D Stereo-IR-Thermo-Laparoscopic Surgical Robot System 1 shown in FIGS. 5A and 5B. As shown, the system is configured with the powered fat tissue aspiration cannula instrument 4, 3D-stereoscopic/IR-thermographic camera 2, and grippers held and controlled by a robotic navigation subsystem 210 that handles the other surgical instruments depicted in FIGS. 2 and 3, while the surgical robot interface control console 200 is manned by a human surgeon.

FIG. 6B shows a global AR-VR (Metaverse)-based surgical system network of the present invention 100 that supports the various systems and subsystems disclosed herein on its internet infrastructure 103 including the 3D-stereoscopic IR-thermographic endoscopic visceral fat tissue aspiration system 1 configured for providing the real-time cytokine detection, analysis, and AR-based surgical display capabilities of the type and kind described herein. As shown in FIG. 6B, the system network 100 comprises: (i) a plurality of GNSS satellites 101A-101N transmitting GNSS signals towards the earth and objects below; (ii) a plurality of GNSS receiver installed within an operating environment 30 for receiving and processing transmitted GNSS signals during monitoring using time averaging seismic data extraction processing; (iii) an internet gateway 107 providing access to the Internet communication infrastructure 103; (iv) one or more client computing systems 109 for transmitting instructions and receiving alerts and notifications and supporting diverse administration, operation and management functions on the system network; (vi) a cell tower 110 for supporting cellular data communications across the system network 100; and (vii) a data center 112 supporting web servers 112A, application servers 112B, database and datastore servers 112C, and SMS/text and email servers 112D.

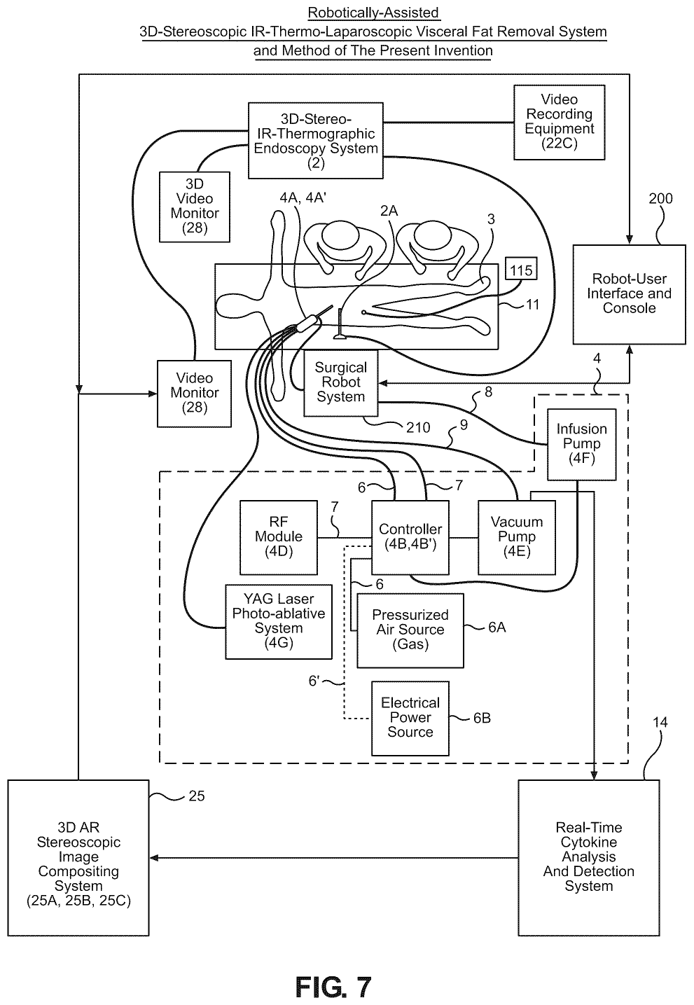

FIG. 7 shows the operating room layout of FIG. 6, with the Robotically-Assisted 3D Stereoscopic IR-Thermographic Laparoscopic Surgical (Robotic) System 1′ of FIGS. 5A and 5B, configured with the powered fat tissue aspiration cannula instrument 4 held and controlled by a robotic navigation subsystem 210 that handles the other surgical instruments depicted in FIGS. 2 and 3.

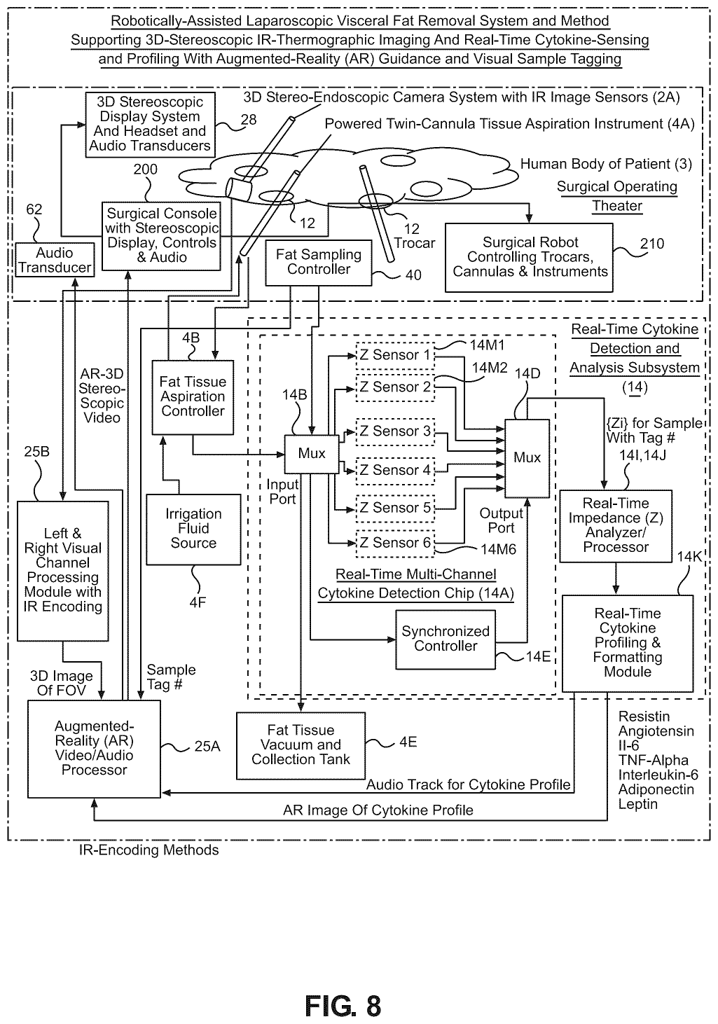

FIG. 8 show the components of the 3D Stereoscopic IR-Thermographic Laparoscopic Visceral Fat Removal System 1 illustrated in FIGS. 5A, 5B, 5C, 6 and 7, also supporting (i) a 3D stereoscopic endoscopic digital camera system 2 with IR image sensors 26, (ii) a powered twin-cannula tissue aspiration instrument system 4, and (iii) a real-time cytokine detection and analysis subsystem 14, and profiling using augmented-reality (AR) guidance and visual sample tagging methods. Using the system 1 shown in FIG. 8, bariatric and like surgeons can (i) safely and effectively sample visceral fat tissue from the mesentery of patients during out-patient treatment using the twin-cannula instrument 4, (iii) automatically tag and index such samples to digital images of the mesentery region from the samples that were obtained, (iii) automatically detect and analyze the specific cytokine concentration of the samples in real-time using the cytokine detection and analysis system 14, and (iv) then display AR-augmented digital images of the sampled region, with cytokine content indices composited with the digital images using AR-display techniques, to provide both visual guidance and support intelligence to the surgeon during obesity treatment operations conducted within the operating room.

### Specification of the 3D-Stereographic/IR-Thermographic Display System of the Present Invention

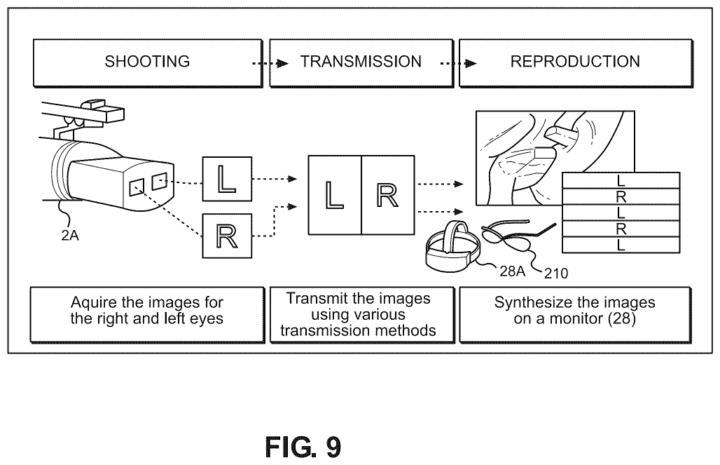

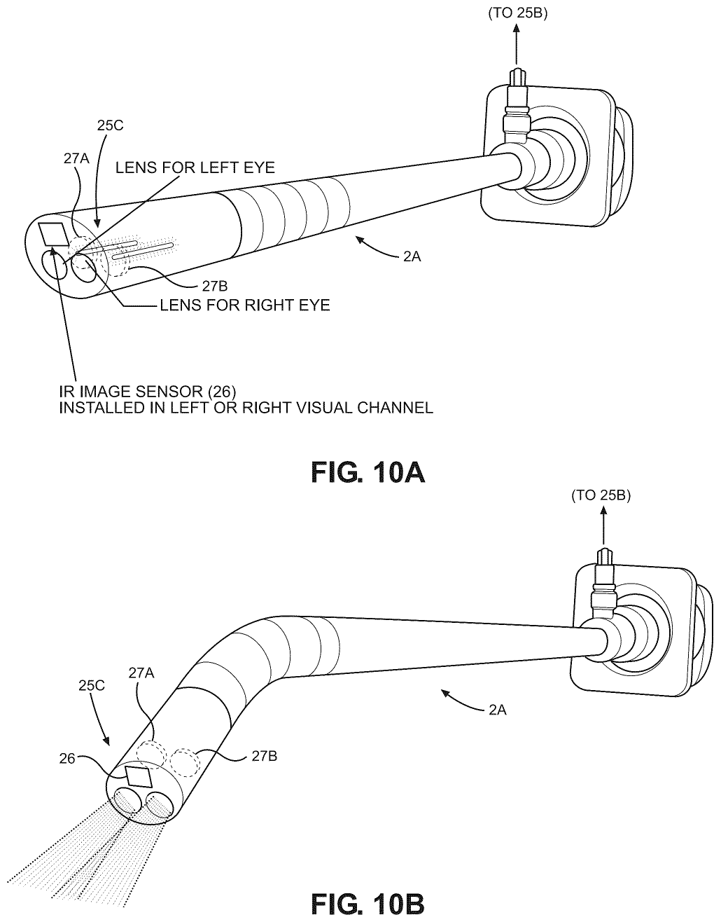

FIGS. 9, 10A and 10B shows the 3D-stereoscopic IR-thermographic laparoscopic digital camera system 2 of the present invention equipped with left and right perspective video image sensors 27A and 27B mounted behind image formation optics mounted in the distal portion of the instrument 2A, for use in generating stereoscopic video images (L/R) for use in 3D stereoscopic viewing of the patient's abdominal region during visceral fat aspiration using the 3D-stereoscopic IR-thermographic laparoscopic visceral fat removal system 1 of the present invention, shown in FIGS. 5A, 5B, 5C, 6 and 7.

FIGS. 10A and 10B show the 3D-stereoscopic IR-thermographic endoscopic digital camera system 2, in which at least one IR image sensor(s) 26 is embedded in at least one visual channel of the 3D-Stereoscopic IR-Thermographic Laparoscopic Visceral Fat Removal System 1 shown in FIGS. 5A, 5B, 5C, 6 and 7. As will described in greater detail hereinafter, the IR thermographic video images from IR camera subsystem 26 will capture the thermal energy (i.e. surface temperature) of tissues within the patient, and help the surgeon visually discriminate visceral fat tissue from other blood carrying vessels that should not typically be aspirated during the method of obesity treatment according to the present invention.

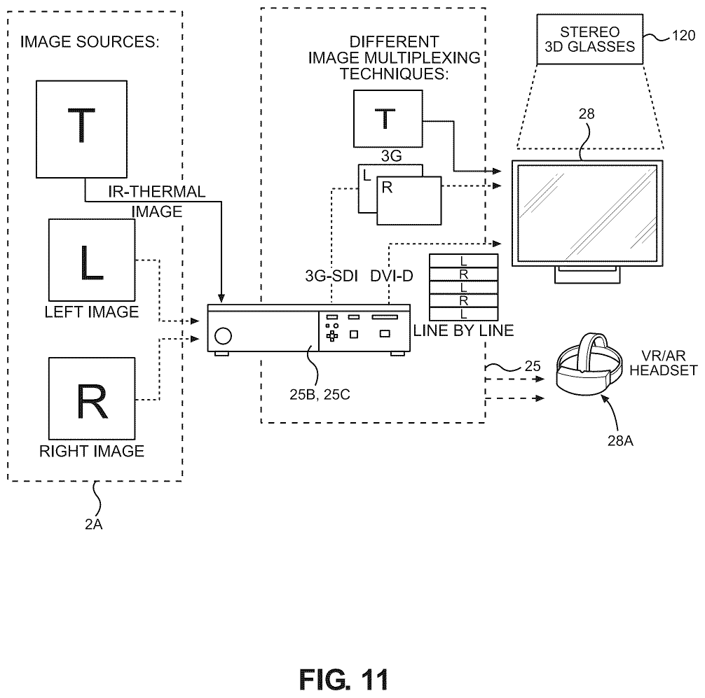

FIG. 11 shows a schematic representation of the 3D-stereoscopic IR-thermographic endoscopic digital camera system 2, depicted in FIGS. 9, 10A and 10B, and employed in the 3D-Stereoscopic IR-Thermographic Laparoscopic Visceral Fat Removal System shown in FIGS. 1A, 1B, 2, 3 and 5A, 5B, 5C, 6 and 7. In general, many different kinds of 3D imaging/display techniques can be supported by the 3D stereoscopic IR-thermographic endoscopic digital camera system 2A, employed within the 3D-stereoscopic IR-thermographic Laparoscopic System 2 for used during visceral fat removal, in accordance with the principles of the present invention. As shown in FIG. 11, polarization eyeglasses or other stereoscopic-decoding eyewear 120, or AR/VR headset 28A shown in FIG. 1C, will be typically worn by the surgeon during surgery so as to stereoscopically-decode (e.g. polarization-decode) left and right perspective images of the surgical field of view that are being displayed on video monitors 28, and thus enable 3D stereoscopic viewing of the patient's mesentery region. When using the AR/VR headset 28A to deliver AR-3D stereoscopic images to the surgeon during surgeon, direct 3D stereoscopic image display methods can be used to deliver left stereoscopic video images to the surgeon's left visual channel, and right stereoscopic video images to the surgeon's right visual channel, while minimizing or eliminating visual channel cross-talk, to provide comfort and reduce surgeon eyestrain.

Specification of the Method of Color-Coding Pixels in IR-Thermographically Captured Surface-Temperature Images of Visceral Fat Tissue Samples Aspirated from within the Mesentery Region of a Patient Using the Soft-Tissue Aspiration Instrumentation of the Present Invention During Surgical Treatment

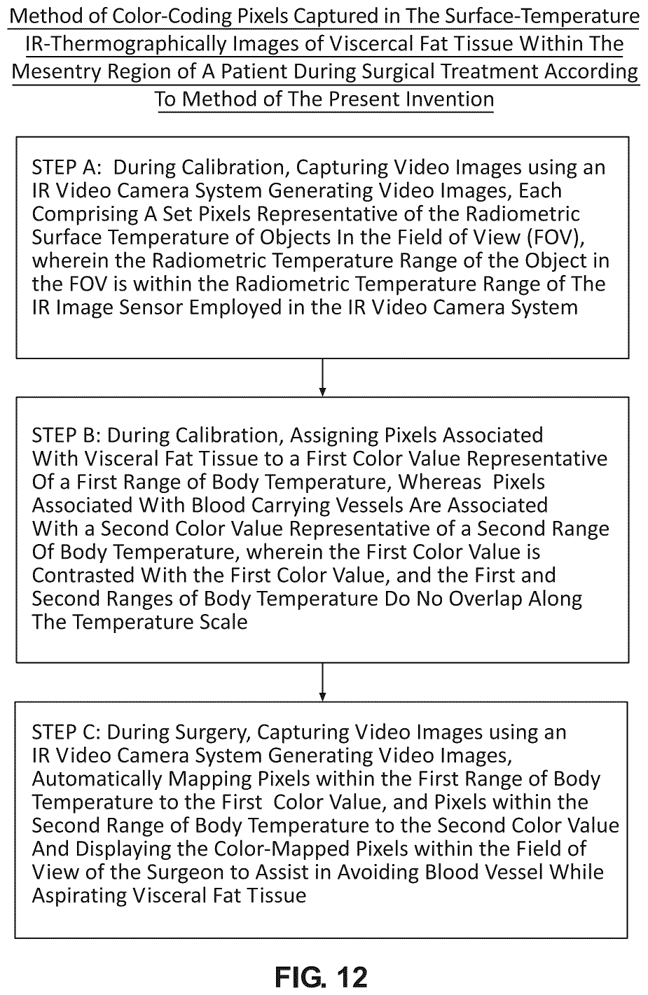

FIG. 12 describes the primary steps involved in the automated method of color-coding pixels captured in the IR-thermographic (surface-temperature) images of visceral fat tissue selectively aspirated from within the mesentery region of a patient using the soft-tissue aspiration instrumentation of the present invention during the method of surgical treatment, illustrated in FIG. 13.

As indicated at STEP A in FIG. 12, the method involves, during calibration, capturing Video Images using an IR video camera system generating video digital images, wherein each digital image comprises a set pixels representative of the radiometric surface temperature of objects in the field of view (FOV), wherein the radiometric temperature range of the object in the FOV is within the radiometric temperature range of the IR image sensor employed in the IR-thermographic video camera system 26 shown in FIGS. 10A and 10B.

As indicated at Step B in FIG. 12, the method involves, during calibration, assigning pixels associated with visceral fat tissue to a first color value representative of a first range of body temperature, whereas pixels associated with blood carrying vessels are associated with a second color value representative of a second range of body temperature, wherein the first color value is contrasted with the first color value, and the first and second ranges of body temperature do no overlap along the temperature scale.

As indicated at Step C in FIG. 12, the method involves, during surgery, capturing video images using an IR video camera system generating video images, automatically mapping pixels within the first range of body temperature to the first color value, and pixels within the second range of body temperature to the second color value and displaying the color-mapped pixels within the field of view of the surgeon to assist in avoiding blood vessel while aspirating visceral fat tissue.

FIG. 13 shows a schematically represented color-coded image of IR-thermographically-imaged visceral fat tissue being selected aspirated from within the mesentery region of a patient undergoing surgical treatment in accordance with the method of the present invention. As shown, the stereoscopic/thermographic display apparatus of the present invention generates and displays (i) color-coded 3D-stereoscopic images of the surgical field of view (FOV) within the patient's mesentery or abdominal region captured by the 3D stereoscopic camera pair 25C1 and 25C2 within the instrument shown in FIGS. 10A, 10B, as well as (ii) (ii) color-coded thermographic images of the surgical field of view within the patient's mesentery or abdominal region captured by the IR thermographic camera pair 26 within the instrument shown in FIGS. 10A and 10B. These two modes of imaging and display can be used individually, as well combined into a third hybrid composited mode to overlay the IR thermographic image over the 3D stereographic images, to enjoy the benefits that thermography provides when used alone or in combination with stereoscopy. These visual display modes will help a surgeon visually distinguish arterial vessels, veins and fat, and facilitate visualization of arterial blood vessels, portal and systemic veins and fat, during surgery.

Preferably, the color-encoding/mapping scheme applied to the captured thermographic video images will be selected so that a range of surface temperature associated with warmer blood flow and circulation will be automatically assigned/mapped to a markedly distinguishable color value, different from the tissue regions having cooler surface temperatures, indicative of less blood flow and/or circulation. This will ensure high-contrast thermographic images of the surgical field of view during tissue aspiration surgery, thereby reducing the risk of accidental damage and/or injury to surrounding tissue and anatomical structures during the obesity treatment procedures.

### Specification of the Visceral Fat Photo-Ablation and Aspiration Subsystem Supported by Laser Photo-Ablation at Point of Aspiration

FIG. 14 shows the twin-cannula bipolar electro-cauterizing, visceral fat photo-ablating and aspirating instrumentation system of the present invention 4, depicted in the system of FIG. 1. As shown, the system 1 comprises: (i) a hand-supportable fat aspiration instrument 4A having (i) a hand-supportable housing 15 with a stationary tubing connector 16 provided at the rear of the housing and receiving a length of flexible tubing 9B connected to a vacuum source 4E and connecting to the cylindrical cannula base portion guide tube 20; and a twin tumescent-type cannula assembly 5 having an inner cannula 5B coupled to an electrically-powered cannula drive mechanism disposed within the hand-supportable housing 15 and preferably powered by a source of electrical power, while its stationary outer cannula 5A is releasably connected to the front portion of the hand-supportable housing 15, and (ii) a system controller 4B for controlling the electro-cautery, irrigation and YAG-laser driven photo-ablative functions supported by the fat aspiration instrument system 4.

FIG. 15A shows the photo-ablative electro-cauterizing fat aspiration instrument system 4 depicted in FIGS. 14 and 15B, showing its components disassembled.

FIG. 15B shows the electromagnetically-actuated fat tissue photo-ablation and aspiration instrument 4 shown in FIGS. 14 and 15A, having a twin-cannula assembly 5 supporting three-functions (i.e. tumescent infusion, electro-cautery and YAG-laser photo-ablation) about the aspiration aperture 8 during visceral fat aspiration operations.

FIGS. 16A and 16B show the distal (tip) portion of the outer cannula component 5A of the twin-cannula assembly 5, illustrating its optical fiber 13 carrying the YAG-laser photonic-energy laser beam 33 to the field about the outer aspiration aperture 8 to support photo-ablative operations on visual fat tissue present within the field, and an irrigation channel 36 for conducting irrigation fluid to the bullet tip area of the outer cannula 5A where aspiration and photo-ablative operations are carried out within the field. When the YAG laser beam is delivered to the field, the visceral fat tissue present therein is automatically photo-ablated while irrigation fluid and remaining tissue is aspirated away down the cannula into the in-line cytokine detection and analysis system 14 for selective cytokine detection, analysis and collection.

In order to optimize/increase/facilitate the intraluminal flow as well as aperture avulsion of fat from vascular pedicles/stalks, the tissue aspiration instrument of the present invention can further include the use of harmonic (e.g. vibratory) pulsation both (i) during vacuum aspiration of visceral fat tissue through aspiration aperture 8 at distal end of the twin cannula assembly, and also (ii) during trocar maintenance of intra-abdominal pressure during the tissue aspiration operations.

Further, a transducer tip can be added to the distal end of the tissue aspiration instrument 4 shown in FIG. 16A to deliver energy that will facilitate the “popping”, emulsification, dissolution, and/or membrane disruption of fat globules within the mesentery region of the patient, along with concurrent supply of cooled saline solution at the distal tip region to prevent adjacent tissue damage during tissue aspiration operations.

Specification of the In-Line Visceral Fat Tissue Analysis Subsystem with Real-Time Cytokine Detection, Analysis and Digital Image Processing

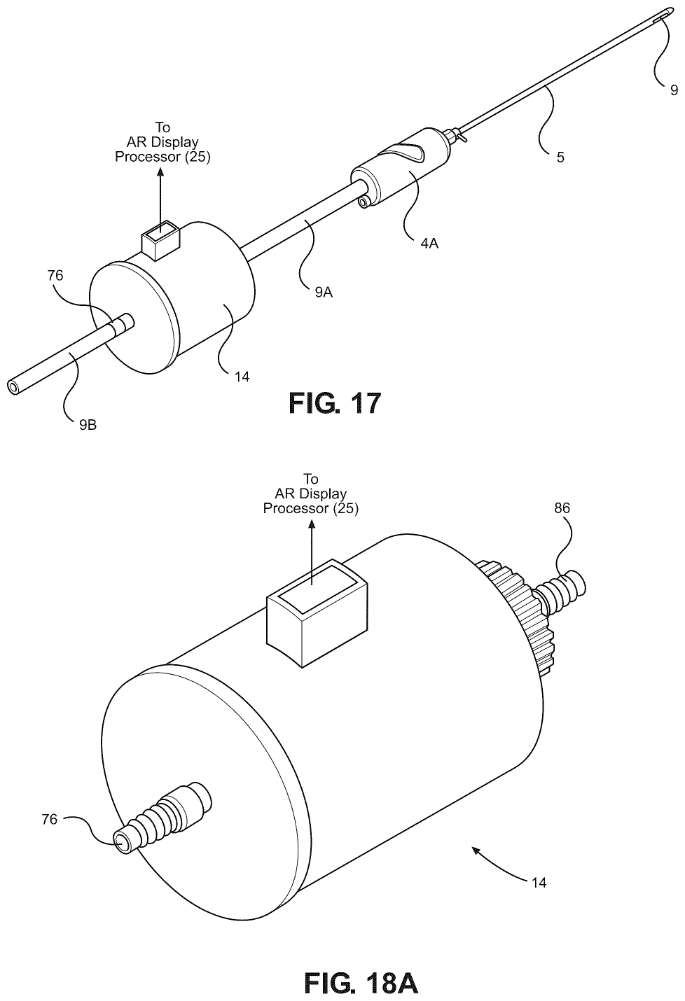

FIG. 17 shows the in-line fat sampling and real-time cytokine detection and analysis device of the present invention 14 depicted in FIGS. 3, 4, 7 and 8, and shown operably connected between a vacuum source 4E and the visceral fat tissue aspiration instrument of the present invention 4 shown in FIGS. 3 and 7.

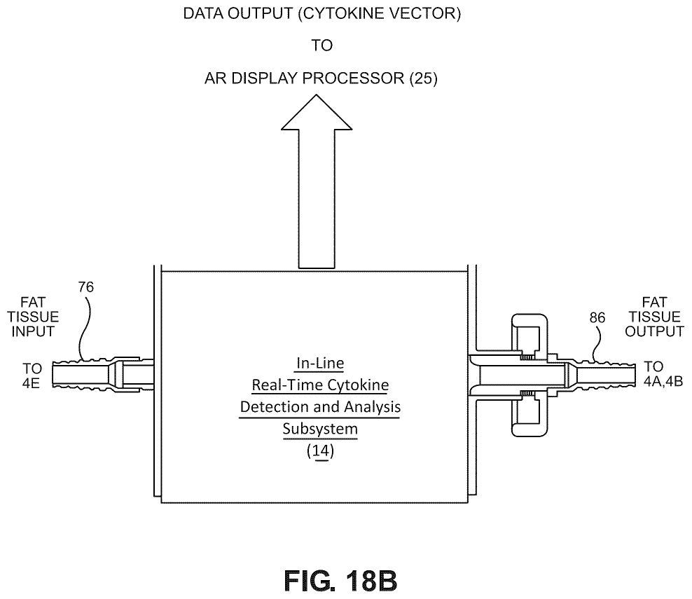

FIG. 18A shows a preferred mechanical construction for the in-line fat sampling and real-time cytokine detection and analysis device 14 depicted in FIGS. 3, 4, 7, 8, and 12.

FIG. 18B shows a cross-sectional schematic construction of the in-line fat sampling and real-time cytokine detection and analysis device 14 depicted in FIGS. 17 and 18A. As shown clearly in 18B, visceral fat tissue aspirated from the patient's mesentery region by the twin-cannula tissue aspiration instrument system 4 is supplied as input to the cytokine detection and analysis system 14, in which the cytokine detection and analysis chip device 4A of FIGS. 3A, 3B and 3C is installed. After automated real-time cytokine detection and analysis processing by the chip device 4A, the aspirated tissue is provided as output for collection and disposal, while the cytokine content information (i.e. cytokine state vector assigned to each processed tissue sample) is automatically abstracted by the system and encoded onto AR 3D stereoscopic display images by the AR display processor 25 for stereoscopic display and visualization by the surgeon during visceral tissue aspiration operations on the patient.

### Method Of Metabolic Treatment Of The Present Invention

FIG. 19 shows a color-coded image of IR-thermographically imaged visceral fat tissue aspirated from within the mesentery region of a patient undergoing surgical treatment in accordance with the principles of the present invention. As shown, the color-coded image is not only pixel-color mapped as described above, but also composited with an augmented-reality (AR) display of the real-time measurement of the cytokine concentration detected in the aspirated visceral fat tissue sample, during a specific date/time-stamped sampling interval of a surgical treatment procedure of the present invention. FIG. 19 also provides an exemplary perspective view of the patient's abdominal region during the mesenteric visceral fat aspiration procedure of the present invention, showing various operations which will typically include: (i) insertion of a bipolar electro-cauterizing twin-cannula tissue aspiration instrument 4 into the mesentery region for infusion of tumescent solution; (ii) insertion and positioning of the twin-cannula visceral fat aspiration instrument 4 into the mesentery region for fat removal by way of visceral fat aspiration under 3D-stereo/IR-thermographic laparoscopic guidance; and (iii) the aspiration and electro-cauterization of visceral fatty tissue in the mesentery using the bipolar electro-cauterizing twin-cannula visceral fat aspiration instrumentation 4.

By removing cytokine-rich visceral fat tissue from a patient's mesentery region using the instruments and methods of the present invention, a number of positive metabolic effects and benefits can be expected, namely: (i) there will be an increased negative feedback effect (i.e. decrease in fat burn) which an increase in hypertropic visceral fat cells will have upon the basal metabolic rate (BMR) within a human being's metabolism, by the increased secretion of Leptin, Resistin and TNF-α—prior to treatment according to the principles of the present invention; (ii) there will be a decreased secretion of cytokines (i.e. Adipopectin) in response to an increase in hypertropic visceral fat cells, favoring a decrease in sensitivity of peripheral tissues to insulin and thus a decrease in glucose utilization thereby—prior to treatment according to the principles of the present invention; (iii) there will be a reduction in the number of hypertropic fat cells and their harmful secretions (i.e. Leptin, Resistin and TNF-α) by the method of treatment according to the present invention, and the favorable impact on the patient's metabolism by increasing fat burn and the basal metabolic rate (BMR); and (iv) there will be an increased circulation of secretion of cytokines (i.e. Adipopectin) in response to a decrease in hypertropic visceral fat cells by practicing the method of treatment according to the present invention, and the favorable increase in sensitivity of peripheral tissues to insulin and thus an increase in glucose utilization thereby.

### Alternative Embodiments Which Readily Come To Mind

While the various methods of 3D-sterescopic/IR-thermoscopic imaging and display, and automated real-time cytokine detection, analysis and AR-display have been illustrated in the context of providing surgical intelligence and guidance during visceral fat removal within obese patients during outpatient procedures, it is understood that such methods and apparatus can also be used with other modes of surgical tissue removal and/or treatment within the body of a patient during other kinds of endoscopic and laparoscopic procedures.

Such image processing and display methods may be used in conjunction with surgical instruments and methods not involving visceral fat tissue. Instead, the methods and instruments can be used in tissue aspiration/sampling and real-time in-flow detection of specific biomarkers for cancers (e.g. PSA for prostate cancer, CEA for colon cancer, and CA125 for ovarian cancer) during surgical exploration, or biopsies, to provide quick feedback to surgeons during surgery regarding the status of whether or not they have attained clear margins of operative resection of a cancerous region in the body of a patient.

In the illustrative embodiments described above, the electrode surfaces of the microchannel sensors 14C within the cytokine detection and analysis chip 14A are automatically coated with cytokine antibody material during each automated impedance measurement preparation process. The purpose of coating the electrode surfaces with cytokine antibody material is to maximize the collection of cytokine protein molecules on the electrodes, during electrical impedance measurements, employed to automatically (i) detect specific types of cytokines present within aspirated fat samples being analyzed in the pipeline of microchannels, and (ii) quantify the specific concentration thereof against known reference measures used by the system. Applying cytokine antibody coatings on microchannel electrode surfaces greatly improves the sensitivity of the cytokine detection instrument system, but this feature also requires conducting additional preparatory steps outlined in FIG. 3B which can add complexity and complications. However, in an alternative embodiment of the present invention, it is taught to eliminate the step of precoating microchannel electrode surfaces with cytokine antibody material, and simply detect the presence of cytokine proteins suspended within the microchannels in a direct manner, using one or more known sensing methods including, but not limited to: electrical impedance measurement using electrical current/voltage sensing; spectroscopic measurement and characterization using photonic/light sensing principles; optical measurement and characterization using light refraction, diffraction, reflection and/or absorption principles; electromagnetic resonance principles; and other techniques known in the material sensing and characterization art that can be used to detect the presence of specific cytokine proteins in solution within the microflow channel, when closed off during detection and measurement operations.

While manually-controlled and robotically-controlled/assisted methods of visceral tissue removal have been described in detail in the illustrative embodiments, local and remote methods of surgical control and management have also been described so that the methods and apparatus of the present invention can be practiced in virtual environment employing AR/VR techniques in a surgical metaverse developing around the planet, as reflected in FIG. 6B. As such, the present invention can be practiced in this new metaverse of human activity beyond surgical treatment, including purposes such as teaching, conducting workshops, and recording educational sessions in a metaverse designed to instruct and educate.

Several modifications to the illustrative embodiments have been described above. It is understood, however, that various other modifications to the illustrative embodiment of the present invention will readily occur to persons with ordinary skill in the art. All such modifications and variations are deemed to be within the scope and spirit of the present invention as defined by the accompanying Claims to Invention.

1-22. (canceled)

23. A 3D-stereoscopic IR-thermographic laparoscopic visceral fat removal system comprising:
a 3D stereoscopic laparoscopic camera system having a pair of digital cameras supporting left and right fields of view for generating stereoscopic image pairs, and one or more IR image sensors mounted along at least one of said left and right fields of view to generate an IR thermographic image;
a 3D stereoscopic display system selectively displaying 3D stereoscopic images of the patient's mesentery region, and IR thermographic images of the patient's mesentery region with or without color mapping of pixels in said IR thermographic images; and
a powered tissue aspiration instrument in communication with a fat tissue vacuum source and collection tank, for selectively aspirating visceral fat tissue from a patient's mesentery region while viewing 3D stereoscopic images of the patient's mesentery region, and/or IR thermographic images of the patient's mesentery region.

24. The 3D-stereoscopic IR-thermographic laparoscopic visceral fat removal system of claim 23, which further comprises:
a cytokine detection and analysis device operably connected to said powered tissue aspiration instrument for automatically detecting and analyzing the cytokine concentration in aspirated fat tissue samples provided as input to said cytokine detection and analysis device, and generating cytokine content data for compositing with said 3D stereoscopic images of the patient's mesentery region, and/or IR thermographic images of the patient's mesentery region, so as to provide augmented reality (AR) images for display to the surgeon.

25. The 3D-stereoscopic IR-thermographic laparoscopic visceral fat removal system of claim 24, which further comprises:
a multiplexer at its input fluid port,
multiple sensor channels through which heated aspirated fat samples flow toward the output fluid port under controlled pressure, while being electrically analyzed across the sensing channel,
a real-time electrical impedance analyzer and processing module,
a real-time cytokine profiling and formatting module, interfaced with a video processor, for displaying detected concentrations of specific cytokines in each tagged sample of visceral fat sampled and aspirated from a particular patient's abdominal region.

26. A cytokine detection and analysis chip comprising:
(i) a micro-fluidic detection chip module, wherein each sensing channel has an input micro-fluidic inflow gate operably connected to the powered aspiration instrument inserted and guided within the patient, and also connected to a micro-fluidic flow detection channel that is connected to an output microfluidic outflow gate, that is operably connected to a flow channel vacuum source;
(ii) a flow channel flushing agent module in fluid communication with each micro-fluidic flow detection channel, for flushing the channel with an appropriate flushing agent under the control of the system controller;
(iii) a flow channel liquid cytokine binding agent module in fluid communication with each micro-fluidic flow detection channel, for supplying the channel with an appropriate cytokine binding agent under the control of the system controller, suited for the particular cytokine to be detected within this specific flow channel on the chip;
(iv) an ultrasonic-based electrode cleaning module in fluid communication with each micro-fluidic flow detection channel, for ultrasonically cleaning the channel with ultrasonic energy and applied fluid under the control of the system controller;
(v) a flow channel impedance measuring module in fluid communication with each micro-fluidic flow detection channel, for electrically measuring the impedance (Z) characteristics of the fat sample flowing within the sensing channel under the control of the system controller,
(vi) an impedance data processing module in communication with the flow channel impedance measuring circuit module, for processing the electrical data collected by the impedance measuring circuit under the control of the system controller;
(vii) a cytokine detection module in communication with the impedance data processor for processing the data and detecting the type and quantity (i.e. concentration) of cytokine molecules in the specific flow sensing channel aboard the chip device, for providing digital output to the AR display controller while it is receiving captured digital image frames and indices during the time of fat sampling, and compositing the data input to generate a composite video output for visual display on the 3D stereoscopic IR display system.

27-31. (canceled)

32. A surgical system network that supports various systems on an internet infrastructure, comprising:
a powered visceral fat tissue aspiration cannula instrument held and controlled by a surgical robotic system that handles other surgical instruments, by way of a remotely situated surgeon sending data commands across a wireless communication network to said surgical robotic system.

33. The surgical system network of claim 32, wherein said other surgical instruments includes an IR-thermographic endoscopic visceral fat tissue aspiration system.

34. The surgical system network of claim 33, wherein said other surgical instruments includes a real-time cytokine detection system with AR-based surgical display capabilities for displaying cytokine concentration data on images of the surgical field of view, captured by an endoscopic camera viewing the surgical field of view during a tissue aspiration operation in the mesentery region of a patient.

35. The surgical system network of claim 32, wherein said surgical robot system further comprises:
(i) a 3D stereoscopic endoscopic digital camera system provided with left and right stereoscopic imaging cameras for capturing left and right stereoscopic image pairs of objects within a surgical field of view, and one or more IR-thermographic image sensors disposed in the field of view, for capturing and displaying IR-thermographic images of objects within the surgical field of view;
(ii) a powered tissue aspiration instrument system for aspirating visceral fat tissue within the surgical field of view; and
(iii) a real-time cytokine detection and analysis subsystem, for real-time analysis and detection of cytokine profiling of aspirated tissue, and display images of the aspirated tissue with real-time cytokine concentration content on the display using augmented-reality (AR) guidance and visual sample tagging methods.

36-49. (canceled)
